(19) United States
(12) Patent Application Publication
(10) Pub. No.: US 2004/0192625 A1
(43) Pub. Date: Sep. 30, 2004

(54) Adrenergic receptor overexpression in airway tissues for the treatment of airway obstructive diseases

(76) Inventor: Stephen Bryant Liggett, Cincinnati, OH (US)

Correspondence Address: Frost Brown Todd, 2200 PNC Center, 201 East Fifth Street, Cincinnati, OH, 45202, US

(21) Appl. No.: 10/221,332

(22) PCT Filed: Mar. 9, 2001
(86) PCT No.: PCT/US01/07522

Related U.S. Application Data
(62) Division of application No. 60/188,404, filed on Mar. 10, 2000.

(51) Int. Cl. A61K 48/00; C12N 15/85

(52) U.S. Cl. 514/44; 435/455

(57) ABSTRACT

The present invention provides methods to protect a subject from a respiratory disorder involving an airway obstructive disease such as asthma or chronic obstructive pulmonary disease. Provided are methods to protect a subject from an airway obstructive disease using gene therapy. β₂-adrenergic receptors (β₂AR) act to relax airway smooth muscle and can serve to counteract hyperresponsiveness. Methods are provided for supplying β₂AR function to cells of the lung and airway, such as smooth muscle and epithelial cells, by β₂AR gene therapy. The β₂AR gene, a modified β₂AR gene, or a part of the gene may be introduced into the cell in a vector such that the gene remains extrachromosomal or may be integrated into the subjects chromosomal DNA for expression. These methods provide for administering to a subject in need of such treatment a therapeutically effective amount of a β₂AR gene, or pharmaceutically acceptable composition thereof, for overexpressing the β₂AR gene. Such methods of expressing the administered β₂AR gene in the lungs and airway provide for: (1) preventing or alleving bronchial hyperresponsiveness; (2) preventing or alleving of an airway obstructive disease, e.g., bronchial hyperreactivity, airway hyperresponsiveness, asthma or chronic obstructive pulmonary disorder (“COPD”); (3) reducing the airway resistance response to inhaled natural or synthetic bronchoconstrictors or allergens or to exercise; and (4) enhancing responsiveness (relaxation) of airway tissues to β-agonists.

This invention was made in part with Government support under Grant Nos. HL45967 and HL41496, awarded by the National Institutes of Health. The Government may have certain rights in this invention.

### FIELD OF THE INVENTION

The present invention is related to methods to protect a subject from a disease involving a respiratory disorder, in particular, a respiratory disorder involving an airway obstructive disease such as asthma or chronic obstructive pulmonary disease. More specifically, the present invention is related to methods to protect a subject from an airway obstructive disease using gene therapy to overexpress a β₂-Adrenergic Receptor gene in the lung and airway tissues of the subject.

### BACKGROUND OF THE INVENTION

Diseases involving inflammation are characterized by the influx of certain cell types and mediators, the presence of which can lead to tissue damage and sometimes death. Diseases involving inflammation are particularly harmful when they afflict the respiratory system, resulting in obstructed breathing, hypoxemia, hypercapnia and lung tissue damage. Obstructive diseases of the airways arc characterized by airflow limitation (i.e., airflow obstruction or narrowing) due to constriction of airway smooth muscle, edema and hypersecretion of mucous leading to increased work in breathing, dyspnea, hypoxemia and hypercapnia. While the mechanical properties of the lungs during obstructed breathing are shared between different types of obstructive airway disease, the pathophysiology can differ.

The most common symptoms of asthma are breathlessness and chest tightness; wheezing, dyspnea, and cough also are prominent. Reduced pulmonary function typical of obstructive rather than restrictive airway disease is usually observed. Asymptomatic periods often alternate with paroxysms.

Of the known triggers of asthma, allergens and exercise have received the most attention. Both are powerful, naturally occurring stimuli; exercise is a potential factor in the daily life of every asthmatic, whereas allergens only affect some persons with asthma. Nevertheless, more is known about the effects of antigen.

Asthma is a chronic inflammatory disorder of the airways in which airflow obstruction occurs due to an active constriction of airway smooth muscle of the bronchi and airway mucous accumulation. Bronchial smooth muscle cells express numerous G protein coupled receptors that modulate contractility including β₂-adrenergic receptors (B₂AR) which act to relax, and muscarinic receptors that act to contract, the muscle. Agonists to the former, and antagonists to the latter, receptors are utilized clinically for reversal of bronchoconstriction. The propensity for airway smooth muscle to constrict in asthma has been termed airway hyperresponsiveness. Thus a hallmark physiological finding in patients with asthma is hyperresponsiveness of the bronchi to inhalation of constrictive agents such as the muscarinic agonist methacholine. This constrictive response in asthmatics is thought to be ultimately due to sensitization of pathways, such as the cholinergic system, that culminate in airway smooth muscle contraction. Typically, nonasthmatics have no detectable airway response to inhalation of methacholine and thus airway hyperreactivity has become a defining physiologic parameter.

Beta agonists are useful as bronchodilator agents; they stimulate beta₂-adrenergic receptors, increase intracellular cAMP, and may inhibit the release of mast cell mediators. Other useful drugs include theophylline and related xanthine drugs, which produce bronchodilation through unknown mechanisms; the biscromone, cromolyn, which prevents the release of mediator substances and blocks respiratory neuronal reflexes, and corticosteroids, which primarily decrease inflammation and edema. Anticholinergic drugs may relieve bronchospasm by blocking parasympathetic cholinergic impulses at the receptor level. Antihistamines occasionally prevent or abort allergic asthmatic episodes, particularly in children, but they can only be partially effective in asthma because histamine is only one of many mediators.

Bronchial hyperreactivity (or airway hyperresponsiveness) is a hallmark of asthma and is closely related to underlying airway inflammation. Worsening of asthma and airway inflammation is associated with increase in bronchial hyperreactivity, which can be induced by both antigenic and non-antigenic stimuli. Although inhaled corticosteroids are relatively safe in adult patients with asthma, these agents have tremendous toxicity in children, including adrenal suppression and reduced bone density and growth. Thus, the search for safer and effective agents that diminish bronchial hyperreactivity continues.

β₂AR are cell surface receptors that couple to the stimulatory guanine nucleotide binding protein (Gₛ), activating adenylyl cyclase. Increased intracellular cAMP mediates relaxation of airway smooth muscle by activation of protein kinase A (PKA). PKA acts to phosphorylate myosin light chain kinase, cell surface K⁺ channels, a Na⁺/K⁺ ATPase, phospholamban, and one or more pumps that lead to sarcoplasmic reticulum uptake of Ca²⁺, and to inhibit the production of inositol phosphates. The net effect is a decrease in intracellular Ca²⁺ and phosphorylation of contractile proteins leading to relaxation. The β₂AR subtype is the predominant βAR expressed on bronchial smooth muscle in humans, although there are some conflicting data regarding the role of the β₁AR subtype in bronchodilatation in various other species. Within the airway, luminal epithelial cells also express β₂AR, and some evidence suggests that activation of these receptors contributes to smooth muscle relaxation via an unknown mediator.

The hierarchy of signalling pathways that establishes physiologic bronchomotor tone is not well established. The principle endogenous agonist for airway β₂AR is epinephrine. So, in the absence of significant elevations in circulating epinephrine, adrenergic control of smooth muscle tone may be primarily due to “basal” coupling of receptor to its effector. Chronic exposure to exogenous agonists in the treatment of bronchospasm can result in desensitization of the β₂AR response (tachyphylaxis). Furthermore, the asthmatic inflammatory milieu itself appears to promote a desensitized β₂AR. These issues have promoted the concept that maintenance or augmentation of non-agonist (basal), and agonist-promoted, β₂AR function in the airway could favorably alter myocyte signalling and airway physiology to effectively block hyperresponsiveness. Due to the dynamic nature of β₂AR regulation, such attempts by pharmacologic or genetic means may lead to feedback regulation of the receptor, Gₛ, or other downstream components of the transduction pathway, thus minimizing the impact at the cellular and physiologic level. Such overexpression has also been shown to cause “promiscuous” coupling of receptors to G proteins that are not natively activated in cells with physiologic levels of receptor. Extensive functional coupling of airway smooth muscle β₂AR to Gᵢ could serve to inhibit adenylyl cyclase and possibly promote mitogen activated protein (MAP) kinase activation, adversely affecting cell growth and possibly airway contractility itself. Promiscuous G_{q} coupling would mimic M3 muscarinic receptor activity and act to bronchoconstrict. Finally, data from some studies suggest that there are significant “spare β₂AR” expressed on airway smooth muscle, such that increasing the number or function of these receptors would have no discernible effect on signalling or physiologic function. Similarly, it may be that other components of the pathway leading to relaxation are limiting factors, such that augmentation of βAR-promoted relaxation by increased receptor expression may not be possible.

The current invention now provides methods for overexpressing the β₂AR in the lung and airway cells, e.g., in smooth muscle and epithelial cells, that has profound effects on cellular signalling, smooth muscle function, and bronchial hyperresponsiveness.

### BRIEF DESCRIPTION OF THE FIGURES

FIG. 1. Polynucleotide Sequence. Nucleotide sequence of β₂-Adrenergic Receptor. (SEQ ID NO: 1).

FIG. 2. Polypeptide Sequence. Polypeptide sequence of β₂-Adrenergic Receptor. (SEQ ID NO. 2). Amino acids are numbered from the initiating methionine.

FIG. 3. Ribonuclease protection assays of SMP8-β₂AR transgene expression in the lung. Total cellular RNA (20 μg) was prepared from whole lung homogenates and simultaneously hybridized with a human β₂AR and a mouse β-actin riboprobe. Markers and full-length, undigested probes are on the left. The β-actin band was present in all samples. A protected fragment corresponding to the human β₂AR mRNA was present in transgenic mice but absent in nontransgenic (NTG) littermates. When band density of the β₂AR fragment was normalized for β-actin expression, transgene expression for the two transgenic lines was not significantly different.

FIG. 4. In situ hybridization of SMP8-β₂AR mice and nontransgenic controls. Cryosections of lungs and tracheas were hybridized with a species-specific human β₂AR antisense riboprobe to detect transgene expression. Darkfield photomicrographs are shown. An arrow denotes areas of smooth muscle. (A) Longitudinal section of trachea from a SMP8-β₂AR transgenic mouse shows that the hybridization signals (white dots) are localized exclusively to smooth muscle. (B) A cross-section of lung parenchyma from a SMP8-β₂AR transgenic mouse shows that the transgene was expressed in the smooth muscle of the airways but was absent in the bronchial epithelium and alveoli. (C and D) No specific hybridization signals were present in either the trachea or peripheral lung of nontransgenic mice.

FIG. 5. Isolation and culture of tracheal smooth muscle cells. Primary cultures of tracheal smooth muscle cells were derived from tracheal explants as described in EXPERIMENTAL PROCEDURES. To confirm that the cells were of smooth muscle origin, immunohistochemistry was performed using an anti-(smooth muscle α-actin) antibody and a FITC-conjugated secondary antibody. (A) Phase contrast microscopy of isolated cells. (B) Immunofluoresence of the same cells stained with the anti-(smooth muscle α-actin) antibody. As shown in this representative experiment, >90% of the cells stain positively for smooth muscle α-actin.

FIG. 6. β₂AR expression in primary cultures of tracheal smooth muscle cells. Primary cultures of tracheal smooth muscle cells were derived from tracheal explants as described in EXPERIMENTAL PROCEDURES. βAR density was determined in membranes prepared from nontransgenic (NTG) and SMP8-β₂AR transgenic smooth muscle cells by radioligand binding with [¹²⁵I]CYP. 10 μM propranolol was used to define nonspecific binding. Results represent the mean±SE of four independent experiments for each group.

FIG. 7. cAMP production and adenylyl cyclase activity in isolated tracheal smooth muscle cells. (A) cAMP levels were measured in intact smooth muscle cells that were grown in 24 well plates and treated with either vehicle or various concentrations of isoproterenol for 10 min at 37° C. Reactions were stopped by the addition of HCl, and cAMP was measured by radioimmunoassay. Results are from four independent experiments. (B) Adenylyl cyclase activity was measured in membranes prepared from tracheal smooth muscle cells of transgenic and nontransgenic mice. Reactions were carried out in the presence of the indicated concentrations of isoproterenol for 10 min at 37° C. Detection of [³²P]cAMP produced was determined by column chromatography. Shown is a representative dose response curve. Data for basal and maximal (10 μM isoproterenol) agonist-stimulated activity in pmol/min/mg are summarized in the bar graph (n=4 experiments).

FIG. 8. Ex vivo assessment of airway function in transgenic and nontransgenic mice. Tracheal contractility was measured using ring preparations that were mounted on wires connected to pressure transducers and immersed in organ baths. The absolute contractile force evoked by acetylcholine is shown in (A). In (B) and (C) the relaxation response to isoproterenol is shown. Rings were pre-contracted to the same extent with 10 μM acetylcholine for these studies. The dose-response for the SMP8-β₂AR rings was shifted to the left (ED₅₀˜60 fold less) than nontransgenic rings. See text for mean results. Pretreatment with ICI 118,551 abolished the enhanced isoproterenol sensitivity in SMP8-β₂AR mice, indicating that the effect was specifically due to overexpression of the β₂AR (C). Curves shown represent the mean±SE of data generated from four different mice in each group.

FIG. 9. In vivo measurement of airway hyperreactivity in conscious, unrestrained mice. Airway responsiveness to methacholine was assessed using a rodent whole body plethysmography system to measure Penh (see EXPERIMENTAL PROCEDURES). The mice were first treated with aerosolized PBS to establish baseline function, which was then followed by increasing doses of aerosolized methacholine. On a separate day, the same mice were first pretreated with aerosolized albuterol (0.1 mg/ml) for 20 min. Penh values are reported as percent of the PBS (baseline) value. Bronchoconstriction was greatest in the untreated, NTG control mice (n=12) with a maximal increase in Penh of 558±42%. Pretreatment with albuterol lowered the maximal increase in Penh to 373±33% (p<0.02). The maximum Penh achieved in the transgenic mice (250±26%, n=9) was less than that of both the untreated (p<0.001) and the pretreated NTG mice (p<0.02). When SMP8-β₂AR mice were pretreated with albuterol, no methacholine-induced bronchoconstriction was observed (maximum Penh was 138±11%, p>0.05 compared to baseline).

### SUMMARY OF THE INVENTION

The present invention provides methods to protect a subject from a respiratory disorder involving an airway obstructive disease such as asthma or chronic obstructive pulmonary disease. Provided are methods to protect a subject from an airway obstructive disease using gene therapy. β₂-adrenergic receptors (β₂AR) act to relax airway smooth muscle and can serve to counteract hyperresponsiveness. Methods are provided for supplying β₂AR function to cells of the lung and airway, such as smooth muscle and epithelial cells, by β₂AR gene therapy. The β₂AR gene, a modified β₂AR gene, or a part of the gene may be introduced into the cell in a vector such that the gene remains extrachromosomal or may be integrated into the subjects chromosomal DNA for expression. These methods provide for administering to a subject in need of such treatment a therapeutically effective amount of a β₂AR gene, or pharmaceutically acceptable composition thereof, for overexpressing the β₂AR gene. Such methods of expressing the administered β₂AR gene in the lungs and airway provide for: (1) preventing or alleving bronchial hyperresponsiveness; (2) preventing or alleving of an airway obstructive disease, e.g., bronchial hyperreactivity, airway hyperresponsiveness, asthma or chronic obstructive pulmonary disorder (“COPD”); (3) reducing the airway resistance response to inhaled natural or synthetic bronchoconstrictors or allergens or to exercise; and (4) enhancing responsiveness (relaxation) of airway tissues to β-agonists.

The β₂AR gene or a part of the gene may or may not be integrated (covalently linked) to chromosomal DNA making up the genome of the subject's target cells. The genes may be introduced into the cell in a vector such that the gene remains extrachromosomal. In such a situation, the gene will be expressed by the cell from the extrachromosomal location. The cells may also be transformed where the exogenous DNA has become integrated into the chromosome so that it is inherited by daughter cells through chromosome replication. The gene may be introduced into an appropriate vector for extrachromosomal maintenance or for integration into the host. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of those in the art.

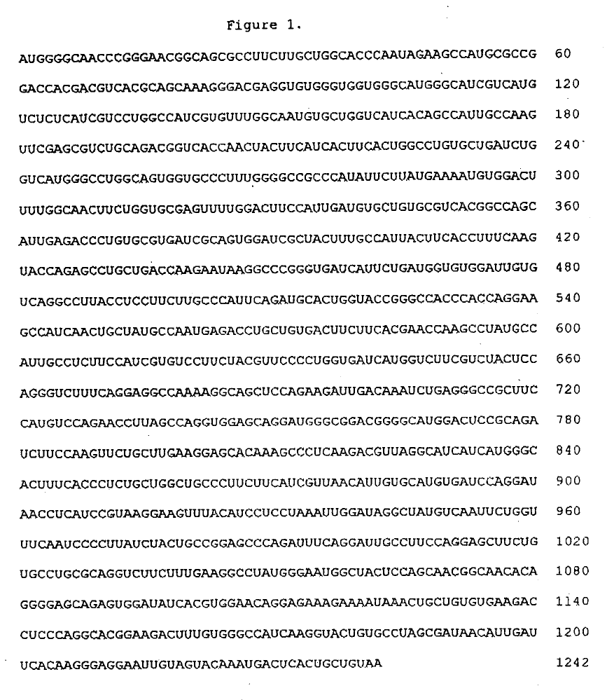

The polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the mature polypeptide may be identical to the coding sequence shown in FIG. 1, (SEQ ID NO:1) or may be a different coding sequence which coding sequence, as a result of the redundancy or degeneracy of the genetic code, encodes the same mature polypeptide as the DNA of FIG. 1.

The polynucleotide which encodes for the mature β₂AR polypeptide of FIG. 1 (SEQ ID NO:2) may include: only the coding sequence for the mature polypeptide; the coding sequence for the mature polypeptide and additional coding sequence; the coding sequence for the mature polypeptide (and optionally additional coding sequence) and non-coding sequence, such as introns or non-coding sequence 5′ and/or 3′ of the coding sequence for the mature polypeptide.

### Definitions

Prior to setting forth the invention, it may be helpful to set forth definitions of certain terms to be used within the disclosure.

The term “airway obstructive disease” refers to a respiratory disorder, such as, airway obstruction, allergies, asthma, acute inflammatory lung disease, chronic inflammatory lung disease, chronic obstructive pulmonary dysplasia, emphysema, pulmonary emphysema, chronic obstructive emphysema, adult respiratory distress syndrome, bronchitis, chronic bronchitis, chronic asthmatic bronchitis, chronic obstructive bronchitis, and intestitial lung diseases.

The term “airway obstruction” refers to an increased resistance to airflow exhibited by characteristic spirometric findings.

The term “chronic bronchitis” refers to the condition associated with prolonged exposure to nonspecific bronchial irritants and is accompanied by mucus hypersecretion and structural changes in the bronchi.

The term “chronic obstructive bronchitis” means the disease condition frequently associated with the symptoms of chronic bronchitis in which disease of the small airways has progressed to the point that there is clinically significant airway obstruction.

The term “pulmonary emphysema” refers to enlargement of the airspaces distal to the terminal nonrespiratory bronchioles, accompanied by destructive changes of the alveolar walls. The term “chronic obstructive emphysema” refers to the condition when there has been sufficient loss Of lung recoil to allow marked airway collapse upon expiration, leading to the physiologic pattern of airway obstruction.

The term “chronic asthmatic bronchitis” refers to an underlying asthmatic condition in patients in whom asthma has become so persistent that clinically significant chronic airflow obstruction is present despite antiasthmatic therapy.

The term “chronic obstructive pulmonary disease or disorder”, or “COPD”, is defined as a generally progressive disease state, due to chronic obstructive bronchitis or chronic obstructive emphysema, which may be accompanied by airway hyperreactivity and may be partially reversible.

Patients not experiencing airflow obstruction, but having chronic bronchitis, emphysema or both, are not included in the definition of COPD. Likewise, those with asthma whose airflow obstruction is completely reversible are not considered to have COPD.

In many cases, however, it is not possible to easily differentiate asthmatic patients with airflow obstruction that does not remit completely, from patients with chronic bronchitis and emphysema who have partially reversible airflow obstruction with airway hyperreactivity. Thus, patients with unremitting asthma are classified as having COPD.

Chronic bronchitis and emphysema with airflow obstruction often occur together and some patients may also have asthma associated with these two disorders.

“Biological activity” refers to a function or set of activities performed by a molecule in a biological context (i.e., in an organism or an in vitro facsimile thereof). Biological activities may include the induction of extracellular matrix secretion from responsive cell lines, the induction of hormone secretion, the induction of chemotaxis, the induction of mitogenesis, the induction of differentiation, or the inhibition of cell division of responsive cells. A recombinant protein or peptide is considered to be biologically active if it exhibits one or more biological activities of its native counterpart.

A “clone” is a population of cells derived from a single cell or common ancestor by mitosis. A “cell line” is a derivative of a primary cell culture that is capable of stable growth in vitro for many generations.

“Deoxyribonucleic Acid (DNA)” is the molecular basis of heredity. DNA consists of a polysugar-phosphate backbone from which the purines and pyrimidines project. Bonds between the phosphate molecule and carbon 3 and carbon 5 of adjacent deoxyribose molecules form the backbone. The nitrogenous base extends from carbon 1 of each sugar. According to the Watson-Crick model, DNA forms a double helix that is held together by hydrogen bonds between specific pairs of bases (thymine to adenine and cytosine to guanine). Each strand in the double helix is complementary to its partner strand in terms of its base sequence.

A DNA “coding sequence” or a “nucleotide sequence encoding” a particular protein, is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences, cDNA from eucaryotic mRNA, genomic DNA sequences from eucaryotic (e.g., mammalian) DNA, and even synthetic DNA sequences. A transcription termination sequence will usually be located 3′ to the coding sequence.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. After digestion, the reaction is electrophoresed directly on an agarose or polyacrylamide gel to isolate the desired fragment.

A “DNA construct” is a DNA molecule, or a clone of such a molecule, either single- or double-stranded that has been modified through human intervention to contain segments of DNA combined and juxtaposed in a manner that as a whole would not otherwise exist in nature.

The term “encoding” refers generally to the sequence information being present in a translatable form, usually operably linked to a promoter. A sequence is operably linked to a promoter when the functional promoter enhances transcription or expression of that sequence. An anti-sense strand is considered to also encode the sequence, since the same informational content is present in a readily accessible form, especially when linked to a sequence that promotes expression of the sense strand. The information is convertible using the standard, or a modified, genetic code.

“Filling” or “blunting” refers to the procedures by which the single stranded end in the cohesive terminus of a restriction enzyme-cleaved nucleic acid is converted to a double strand. This eliminates the cohesive terminus and forms a blunt end. This process is a versatile tool for converting a restriction cut end that may be cohesive with the ends created by only one or a few other restriction enzymes into a terminus compatible with any blunt-cutting restriction endonuclease or other filled cohesive terminus.

The term “gene” means the segment of DNA involved in producing a polypeptide chain; it includes regions preceding and following the coding region “leader and trailer” as well as intervening sequences (introns) between individual coding segments (exons).

The term “intron” identifies an intervening sequence within a gene for the gene product that does not constitute protein coding sequences. In cukaryotic cells introns are removed from the primary RNA transcript to produce the mature mRNA.

An “isolated” nucleic acid is a nucleic acid, e.g., an RNA, DNA, or a mixed polymer, which is substantially separated from other DNA sequences which naturally accompany a native human sequence, e.g., ribosomes, polymerases, and many other human genome sequences. The term embraces a nucleic acid sequence that has been removed from its naturally occurring environment, and includes recombinant or cloned DNA isolates and chemically synthesized analogs or analogs biologically synthesized by heterologous systems. A substantially pure molecule includes isolated forms of the molecule. An isolated nucleic acid will generally be a homogenous composition of molecules, but will, in some embodiments, contain minor heterogeneity. This heterogeneity is typically found at the polymer ends or portions not critical to a desired biological function or activity.

An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein that has been removed from its naturally occurring environment, and includes recombinant or cloned DNA isolates and chemically synthesized analogs or analogs biologically synthesized by heterologous systems.

“β₂AR Allele” refers to normal alleles of the β₂AR locus as well as alleles carrying variations that predispose individuals to develop asthma.

“β₂AR Locus,” “β₂AR Gene,” “β₂AR Nucleic Acids” or “β₂AR Polynucleotide” each refer to polynucleotides, all of which are in the β₂AR region, that are likely to be expressed in normal tissue. The β₂AR locus is intended to coding sequences, intervening sequences and regulatory elements controlling transcription and/or translation. The β₂AR locus is intended to include all allelic variations of the DNA sequence. These terms, when applied to a nucleic acid, refer to a nucleic acid that encodes a β₂AR polypeptide, fragment, homolog or variant, including, e.g., protein fusions or deletions. The nucleic acids of the present invention will possess a sequence that is either derived from, or substantially similar to a natural β₂AR-encoding gene or one having substantial homology with a natural β₂AR-encoding gene or a portion thereof. The coding sequence for a β₂AR polypeptide is shown in SEQ ID NO:1, with the amino acid sequence shown in SEQ ID NO:2.

“β₂AR Region” refers to a portion of human chromosome 5. This region contains the β₂AR locus, including the β₂AR gene. As used herein, the terms “β₂AR locus,” “β₂AR allele” and “β₂AR region” all refer to the double-stranded DNA comprising the locus, allele, or region, as well as either of the single-stranded DNAs comprising the locus, allele or region. As used herein, a “portion” of the β₂AR locus or region or allele is defined as having a minimal size of at least about 200 nucleotides and preferably have a minimal size of at least about 400 nucleotides.

“β₂AR protein” or “β₂AR polypeptide” refer to a protein or polypeptide encoded by the β₂AR locus, variants or fragments thereof. The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, unnatural amino acids, etc.), polypeptides with substituted linkages as well as other modifications known in the art, both naturally and non-naturally occurring. Ordinarily, such polypeptides will be at least about 50% homologous to the native β₂AR sequence, preferably in excess of about 90%, and more preferably at least about 95% homologous. Also included are proteins encoded by DNA that hybridize under high or low stringency conditions, to β₂AR -encoding nucleic acids and closely related polypeptides or proteins retrieved by antisera to the β₂AR protein(s). The β₂AR polypeptide of the present invention also includes conservative variations of the polypeptide sequence. The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine glutamic for aspartic acids, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

A “ligand” is a molecule, other than an antibody or an immunoglobulin, capable of being bound by the ligand-binding domain of a receptor. The molecule may be chemically synthesized or may occur in nature.

“Ligation” refers to the process of forming phosphodiester bonds between two double stranded nucleic acid fragments. Unless otherwise provided, ligation is accomplished using known buffers and conditions with T4 DNA ligase (“ligase”) and approximately equimolar amounts of the DNA fragments to be ligated.

The term “maintained” refers to the stable presence of a plasmid within a transformed host cell wherein the plasmid is present as an autonomously replicating body or as an integrated portion of the host genome.

“Nucleic Acid Hybridization” is a method for using the principle that two single-stranded nucleic acid molecules that have complementary base sequences will reform the thermodynamically favored double-stranded structure if they are mixed in solution under the proper conditions. The double-stranded structure will be formed between two complementary single-stranded nucleic acids even if one is immobilized on a nitrocellulose filter. In the Southern hybridization procedure, the latter situation occurs. The DNA of the individual to be tested is digested with a restriction endonuclease, fractionated by agarose gel electrophoresis, converted to the single-stranded form, and transferred to nitrocellulose paper, making it available for reannealing to the hybridization probe. A “hybridization Probe” is used to visualize a particular DNA sequence in the Southern hybridization procedure using a labeled DNA molecule or hybridization probe that is reacted to the fractionated DNA bound to the nitrocellulose filter. The areas on the filter that carry DNA sequences complementary to the labeled DNA probe become labeled themselves as a consequence of the reannealing reaction. The areas of the filter that exhibit such labeling are visualized. Molecular cloning of a specific DNA sequence from the human genome generally produces the hybridization probe.

“Oligonucleotides” refers to either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The term “open reading frame” refers to a nucleotide sequence with the potential for encoding a protein.

“Operably linked” refers to a juxtaposition wherein the components so described are in a relationship permitting them to function in their intended manner. For instance, a promoter is operably linked to a coding sequence if the promoter affects its transcription or expression.

The term “polylinker” refers a closely arranged series of synthetic restriction enzyme cleavage sites within a plasmid.

The term “plasmid” and “vector” refer to an autonomous self-replicating extrachromosomal circular DNA and includes both the expression and non-expression types. When a recombinant microorganism or cell culture providing expression of a molecule is described as hosting an expression plasmid, the term “expression plasmid” includes both extrachromosomal circular DNA and DNA that has been incorporated into the host chromosome(s).

“Probes”. Polynucleotide polymorphisms associated with β₂AR alleles which predispose to certain cancers or are associated with most cancers are detected by hybridization with a polynucleotide probe which forms a stable hybrid with that of the target sequence, under stringent to moderately stringent hybridization and wash conditions. If it is expected that the probes will be perfectly complementary to the target sequence, stringent conditions will be used. Hybridization stringency may be lessened if some mismatching is expected, for example, if variants are expected with the result that the probe will not be completely complementary. Conditions are chosen which rule out nonspecific/adventitious bindings, that is, which minimize noise. Since such indications identify neutral DNA polymorphisms as well as mutations, these indications need further analysis to demonstrate detection of a B2AR susceptibility allele.

The term “promoter” is a region of DNA involved in binding RNA polymerase to initiate transcription.

The term “protein” is used herein to designate a naturally occurring polypeptide. The term “polypeptide” is used in its broadest sense, i.e., any polymer of amino acids (dipeptide or greater) linked through peptide bonds. Thus, the term “polypeptide” includes proteins, oligopeptides, protein fragments, analogs, muteins, fusion proteins and the like. “Native” proteins or polypeptides refer to proteins or polypeptides recovered from a source occurring in nature. Thus, the term “native leukotoxin” would include naturally occurring leukotoxin and fragments thereof.

“Protein modifications or fragments” are provided by the present invention for β₂AR polypeptides or fragments thereof which are substantially homologous to primary structural sequence but which include, e.g., in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as 32P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods of labeling polypeptides are well known in the art.

Besides substantially full-length polypeptides, the present invention provides for biologically active fragments of the polypeptides or modifications of the polypeptides that could improve efficacy. Significant biological activities include ligand-binding, immunological activity and other biological activities characteristic of β₂AR polypeptides. Immunological activities include both immunogenic function in a target immune system, as well as sharing of immunological epitopes for binding, serving as either a competitor or substitute antigen for an epitope of the β₂AR protein. As used herein, “epitope” refers to an antigenic determinant of a polypeptide. An epitope could comprise three amino acids in a spatial conformation that is unique to the epitope. Generally, an epitope consists of at least five such amino acids, and more usually consists of at least 8-10 such amino acids. Methods of determining the spatial conformation of such amino acids are known in the art.

The term “recombinant” refers to a nucleic acid sequence that is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the common natural forms. Restriction enzyme recognition sites are often the target of such artificial manipulations, but other site-specific targets, e.g., promoters, DNA replication sites, regulation sequences, control sequences, or other useful features may be incorporated by design. A similar concept is intended for a recombinant, e.g., fusion, polypeptide.

“Regulatory sequences” refers to those sequences normally within 100 kb of the coding region of a locus, but they may also be more distant from the coding region, which affect the expression of the gene (including transcription of the gene, and translation, splicing, stability or the like of the messenger RNA).

A “replicon” is any genetic element (e.g., plasmid, chromosome, virus) that functions as an autonomous unit of DNA replication in vivo; i.e., capable of replication under its own control.

A “Restriction Endonuclease” (also called a restriction enzyme) is an enzyme that has the capacity to recognize a specific base sequence (usually 4, 5, or 6 base pairs in length) in a double-stranded DNA molecule, and to cleave both strands of the DNA molecule at every place where this sequence appears. For example, EcoRI recognizes the base sequence GAATTC/CTTAAG.

A “Restriction Fragment” is a DNA molecule produced by digestion with a restriction endonuclease. Any given genome will be digested by a particular restriction endonuclease into a discrete set of restriction fragments.

The term “splice” refers to the removal of an intron from the primary RNA transcript of a gene.

The term “subject” is intended to include living organisms susceptible to airway obstructive disease such as asthma and COPD, particularly mammals. Examples of subjects include humans, dogs, cats, horses, cows, goats, rats and mice. The term “subject” further is intended to include transgenic species.

“Substantial homology or similarity”. A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98%, of the nucleotide bases.

Alternatively, substantial homology or (similarity) exists when a nucleic acid or fragment thereof will hybridize to another nucleic acid (or a complementary strand thereof) under selective hybridization conditions, to a strand, or to its complement. Selectivity of hybridization exists when hybridization that is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. The preparation of such probes and suitable hybridization conditions are well known in the art.

The terms “substantial homology” or “substantial identity”, when referring to polypeptides, indicate that the polypeptide or protein in question exhibits at least about 30% identity with an entire naturally-occurring protein or a portion thereof, usually at least about 70% identity, and preferably at least about 95% identity.

“Substantially similar function” refers to the function of a modified nucleic acid or a modified protein, with reference to the wild-type β₂AR nucleic acid or wild-type β₂AR polypeptide. The modified polypeptide will be substantially homologous to the wild-type β₂AR polypeptide and will have substantially the same function. The modified polypeptide may have an altered amino acid sequence and/or may contain modified amino acids. In addition to the similarity of function, the modified polypeptide may have other useful properties, such as a longer half-life. The similarity of function (activity) of the modified polypeptide may be substantially the same as the activity of the wild-type β₂AR polypeptide. Alternatively, the similarity of function (activity) of the modified polypeptide may be higher than the activity of the wild-type β₂AR polypeptide. The modified polypeptide is synthesized using conventional techniques, or is encoded by a modified nucleic acid and produced using conventional techniques. The modified nucleic acid is prepared by conventional techniques. A nucleic acid with a function substantially similar to the wild-type β₂AR gene function produces the modified protein described above.

Homology, for polypeptides, is typically measured using sequence analysis software. Protein analysis software matches similar sequences using measure of homology assigned to various substitutions, deletions and other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

A polypeptide “fragment,” “portion” or “segment” is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids. The polypeptides of the present invention, if soluble, may be coupled to a solid-phase support, e.g., nitrocellulose, nylon, column packing materials (e.g., Sepharose beads), magnetic beads, glass wool, plastic, metal, polymer gels, cells, or other substrates. Such supports may take the form, for example, of beads, wells, dipsticks, or membranes.

“Target region” refers to a region of the nucleic acid that is amplified and/or detected. The term “target sequence” refers to a sequence with which a probe or primer will form a stable hybrid under desired conditions.

“Transcriptional fusions” are gene fusions in which all coding sequences are derived from the responder gene.

“Translational fusions” are gene fusions that encode a polypeptide comprising coding information of the controller and responder genes.

The term “treatment” means the alleviation of the symptoms of an airway obstructive disease and/or preservation of lung function and/or the general improvement in the patient's perceived quality of life as regards the disease conditions and symptoms.

The term “upstream” identifies sequences proceeding in the opposite direction from expression; for example, the bacterial promoter is upstream from the transcription unit.

A “vector” is a replicon, such as a plasmid, phage, or cosmid, to which another DNA segment may be attached so as to bring about the replication of the attached segment.

A “promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eucaryotic promoters will often, but not always, contain “TATA” boxes and “CAT” boxes. Procaryotic promoters contain Shine-Dalgarno sequences in addition to the −10 and −35 consensus sequences.

DNA “control sequences” refer collectively to promoter sequences, ribosome binding sites, polyadenylation signals, transcription termination sequences, upstream regulatory domains, enhancers, and the like, which collectively provide for the transcription and translation of a coding sequence in a host cell.

A coding sequence is “operably linked to” another coding sequence when RNA polymerase will transcribe the two coding sequences into mRNA, which is then translated into a chimeric polypeptide encoded by the two coding sequences. The coding sequences need not be contiguous to one another so long as the transcribed sequence is ultimately processed to produce the desired chimeric protein.

A control sequence “directs the transcription” of a coding sequence in a cell when RNA polymerase will bind the promoter sequence and transcribe the coding sequence into mRNA, which is then translated into the polypeptide encoded by the coding sequence.

A cell has been “transformed” by exogenous DNA when such exogenous DNA has been introduced inside the cell membrane. Exogenous DNA may or may not be integrated (covalently linked) to chromosomal DNA making up the genome of the cell. In procaryotes and yeasts, for example, the exogenous DNA may be maintained on an episomal element, such as a plasmid. With respect to eucaryotic cells, a transformed cell is one in which the exogenous DNA has become integrated into the chromosome so that it is inherited by daughter cells through chromosome replication. This stability is demonstrated by the ability of the eucaryotic cell to establish cell lines or clones comprised of a population of daughter cell containing the exogenous DNA.

The DNA sequence encoding β₂AR may be cDNA or genomic DNA or a fragment thereof. The term “β₂AR gene” means the open reading frame encoding specific β₂AR polypeptides, as well as adjacent 5′ and 3′ non-coding nucleotide sequences involved in the regulation of expression, up to about 1 kb beyond the coding region, in either direction. The gene may be introduced into an appropriate vector for extrachromosomal maintenance or for integration into the host.

The term “cDNA” as used herein is intended to include all nucleic acids that share the arrangement of sequence elements found in native mature mRNA species, where sequence elements are exons, 3′ and 5′ non-coding regions. Normally mRNA species have contiguous exons, with the intervening introns deleted, to create a continuous open reading frame encoding β₂AR.

### DETAILED DESCRIPTION OF THE INVENTION

β₂-adrenergic receptors (β₂AR) act to relax airway smooth muscle and can serve to counteract hyperresponsiveness. According to the present invention, a method is provided of supplying β₂AR function to cells of the lung and airway, such as smooth muscle and epithelial cells, by β₂AR gene therapy. The β₂AR gene, a modified β₂AR gene, or a part of the gene may be introduced into the cell in a vector such that the gene remains extrachromosomal. In such a situation, the β₂AR gene will be expressed by the cell from the extrachromosomal location.

In accordance with the present invention, there is provided a method of treating airway obstructive disease comprising the administration to a patient in need of such treatment a therapeutically effective amount of a β₂AR gene, or pharmaceutically acceptable composition thereof.

Such methods of providing the β₂AR gene to the lungs and airway provide for:

(1) preventing or alleviating bronchial hyperresponsiveness;

(2) preventing or alleviating of an airway obstructive disease, e.g., bronchial hyperreactivity, airway hyperresponsiveness, asthma or chronic obstructive pulmonary disorder (“COPD”);

(3) reducing the airway resistance response to inhaled natural or synthetic bronchoconstrictors or allergens or to exercise; and

(4) enhancing responsiveness (relaxation) of airway tissues to β-agonists.

### Therapeutic Administration

According to the method of treatment of the present invention, conditions subsumed under the above definition of airway obstructive disease, particularly asthma and chronic obstructive pulmonary disease (COPD) are treated in a patient in need of such treatment by administering to the patient a therapeutically effective amount of a compound of the invention, in such amounts and for such time as is necessary to achieve the desired result. By a “therapeutically effective amount” of a compound of the invention is meant a sufficient amount of the compound to effectively ameliorate the course of the disease and/or alleviate one or more symptoms of airway obstructive disease, or improve the quality of life in a patient at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the severity of the disorder; the activity of the compound employed; the specific composition employed; the age, body weight, general health, sex and diet of the patient; the time of administration, route of administration, and rate of excretion of the compound employed; the duration of the treatment; drugs used in combination or coincidental with the specific compound employed; and like factors well known in the medical arts. Since some of these parameters vary from patient to patient, it is a well-known technique utilized by medical practitioners to determine the proper dose for a particular patient by “dose titrating” the patient; that is, by using the technique of starting with a dose lower than that required to obtain the desired effect, and gradually increasing the dose over time until the desired therapeutic benefit is obtained.

Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of those in the art.

The polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the mature polypeptide may be identical to the coding sequence shown in FIG. 1, (SEQ ID NO:1) or may be a different coding sequence which coding sequence, as a result of the redundancy or degeneracy of the genetic code, encodes the same mature polypeptide as the DNA of FIG. 1.

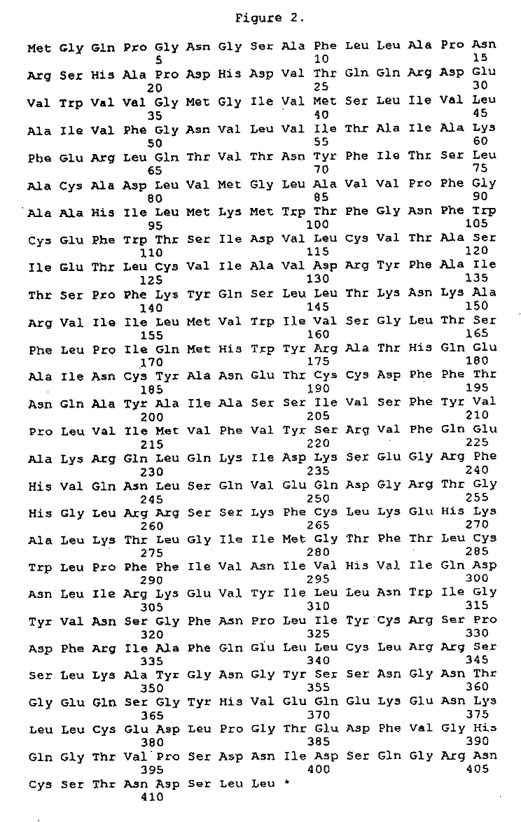

The polynucleotide which encodes for the mature polypeptide of FIG. 2 (SEQ ID NO:2) may include: only the coding sequence for the mature polypeptide; the coding sequence for the mature polypeptide and additional coding sequence; the coding sequence for the mature polypeptide (and optionally additional coding sequence) and non-coding sequence, such as introns or non-coding sequence 5′ and/or 3′ of the coding sequence for the mature polypeptide.

The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

In vivo expression of β₂AR transgenes is preferably by injection of transgenes directly into a specific tissue, such as direct intratracheal, intramuscular or intraarterial injection of naked DNA or of DNA-cationic liposome complexes, or to ex vivo transfection of host cells, with subsequent reinfusion.

Multiple approaches for introducing functional new genetic material into cells, both in vitro and in vivo are known. These approaches include integration of the gene to be expressed into modified retroviruses; integration into non-virus vectors; or delivery of a transgene linked to a heterologous promoter-enhancer element via liposomes; coupled to ligand-specification-based transport systems or the use of naked DNA expression vectors;. Direct injection of transgenes into tissue produces localized expression

PCT/US90/01515 (Felgner et al.) is directed to methods for delivering a gene coding for a pharmaceutical or immunogenic polypeptide to the interior of a cell of a vertebrate in vivo. PCT/US90/05993 (Brigham) is directed to a method for obtaining expression of a transgene in mammalian lung cells following either iv or intratracheal injection of an expression construct.

While most gene therapy strategies have relied on transgene insertion into retroviral or DNA virus vectors, lipid carriers, may be used to transfect the lung cells of the host.

Preparation of Recombinant or Chemically Synthesized Nucleic Acids, Vectors and Host Cells

Large amounts of the polynucleotides of the present invention may be produced by replication in a suitable host cell. Natural or synthetic polynucleotide fragments coding for a desired fragment will be incorporated into recombinant polynucleotide constructs, usually DNA constructs, capable of introduction into and replication in a prokaryotic or eukaryotic cell. Usually the polynucleotide constructs will be suitable for replication in a unicellular host, such as yeast or bacteria, but may also be intended for introduction to (with and without integration within the genome) cultured mammalian or plant or other eukaryotic cell lines.

The polynucleotides of the present invention may also be produced by chemical synthesis and may be performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

Polynucleotide constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate, whether from a native β₂AR protein or from other receptors or from secreted polypeptides of the same or related species, which allow the protein to cross and/or lodge in cell membranes, and thus attain its functional topology, or be secreted from the cell. Such vectors may be prepared by means of standard recombinant techniques well known in the art.

An appropriate promoter and other necessary vector sequences will be selected so as to be functional in the host, and may include, when appropriate, those naturally associated with β₂AR genes. Many useful vectors are known in the art and may be obtained from such vendors as Stratagene, New England Biolabs, Promega Biotech, and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Appropriate non-native mammalian promoters might include the early and late promoters from SV40 or promoters derived from murine Moloney leukemia virus, mouse tumor virus, avian sarcoma viruses, adenovirus II, bovine papilloma virus or polyoma. In addition, the construct may be joined to an amplifiable gene so that multiple copies of the gene may be made.

While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection, or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation.” The cells into which have been introduced nucleic acids described above are meant to also include the progeny of such cells.

Large quantities of the nucleic acids of the present invention may be prepared by expressing the β₂AR nucleic acids or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used.

Mammalian or other eukaryotic host cells, such as those of yeast, filamentous fungi, plant, insect, or amphibian or avian species, may also be useful for production of the proteins of the present invention. Propagation of mammalian cells in culture is per se well known. Examples of commonly used mammalian host cell lines are VERO and HeLa cells, Chinese hamster ovary (CHO) cells, and WI38, BHK, and COS cell lines, although it will be appreciated by the skilled practitioner that other cell lines may be appropriate, e.g., to provide higher expression, desirable glycosylation patterns, or other features.

Clones are selected by using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

The present methods utilize gene therapy methods of expressing or overexpressing the β₂AR peptide sequence “MGQPGNGSAFLLAPNRSHAPDHDVTQQRDEVWVVGMGIVMSLIVLAIVFG NVLVITAIAKFERLQTVTNYFITSLACADLVMGLAVVPFGAAHILMKMWTFG NFWCEFWTSIDVLCVTASIETLCVIAVDRYFAITSPFKYQSLLTKNKARVIILM VWIVSGLTSFLPIQMHWYRATHQEAINCYANETCCDFFTNQAYAIASSIVSFY VPLVIMVFVYSRVFQEAKRQLQKIDKSEGRFHVQNLSQVEQDGRTGHGLRRS SKFCLKEHKALKTLGIIMGTFTLCWLPFFIVNIVHVIQDNLIRKEVYILLNWIGY VNSGFNPLIYCRSPDFRIAFQELLCLRRSSLKAYGNGYSSNGNTGEQSGYHVE QEKENKLLCEDLPGTEDFVGHQGTVPSDNIDSQGRNCSTNDSLL*” as a method of prevention and treatment of airway obstruction disorders.

TABLE 1
Three- and One-Letter Abbreviations (common) for amino acids:

| | |
|---|---|
| Asp = | D = Aspartic Acid |
| Ala = | A = Alanine |
| Arg = | R = Arginine |
| Asn = | N = Asparagine |
| Cys = | C = Cysteine |
| Gly = | G = Glycine |
| Glu = | E = Glutamic Acid |
| Gln = | Q = Glutamine |
| His = | H = Histidine |
| Ile = | I = Isoleucine |
| Leu = | L = Leucine |
| Lys = | K = Lysine |
| Met = | M = Methionine |
| Phe = | F = Phenylalanine |
| Pro = | P = Proline |
| Ser = | S = Serine |
| Thr = | T = Threonine |
| Trp = | W = Tryptophan |
| Tyr = | Y = Tyrosine |
| Val = | V = Valine |

TABLE 2
Conservative Amino Acid Replacements

| | | |
|---|---|---|
| For Amino Acid | Code | Replace With: |

| | | |
|---|---|---|
| Alanine | A | D-Ala, Gly, Aib, B-Ala, Acp, L-Cys, D-Cys |
| Arginine | R | D-Arg, Lys, D-Lys, homo-Arg, D-homo-Arg, |
| | * | Met, Ile, D-.Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, beta-Ala, Acp |
| Isoleucine | I | D-Ile, Val, D-Val, AdaA, AdaG, Leu, D-Leu, |
| | * | Met, D-Met |
| Leucine | L | D-Leu, Val, D-Val, AdaA, AdaG, Leu, D-Leu, |
| | * | Met, D-Met |
| Lysine | K | D-Lys, Arg, D-Arg, homo-Arg, D-homo-Arg, |
| | * | Met, D-Met, Ile, D-Ile, Orn, D-Orn |
| Methionine | M | D-Met, S-Me-Cys, Ile, D-Ile, Leu, D-Leu, |
| | * | Val, D-Val |
| Phenylalanine | F | D-Phe, Tyr, D-Thr, L-Dopa, His, D-His, |
| | * | Trp, D-Trp, Trans-3,4, or 5-phenylproline, |
| | * | AdaA, AdaG, cis-3,4, or 5-phenylproline, |
| | * | Bpa, D-Bpa |
| Proline | P | D-Pro, L-I-thioazolidine-4-carboxylic |
| | * | acid, D-or L-1-oxazolidine-4-carboxylic |
| | * | acid (Kauer, U.S. Pat. No. (4,511,390) |
| Serine | S | D-Ser, Thr, D-Thr, allo-Thr, Met, D-Met, |
| | * | Met(O), D-Met(O), L-Cys, D-Cys |
| Threonine | T | D-Thr, Ser, D-Ser, allo-Thr, Met, D-Met, |
| | * | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | * | AdaA, AdaG |

The methods of the present invention utilize gene sequences encoding a peptide containing at least 100 or more amino acids with at least 70% sequence identity to an amino acid sequence of β₂AR. In preferred embodiments, the methods of the present invention utilize gene sequences encoding a peptide containing at least 200 or more amino acids with at least 70% sequence identity to an amino acid sequence of β₂AR. In more preferred embodiments, the methods of the present invention utilize gene sequences encoding a peptide containing at least 300 or more amino acids with at least 80% sequence identity to an amino acid sequence of β₂AR and the amino acid sequence of β₂AR is the amino acid sequence of peptide of Sequence No. 2 from about position 34 to about position 341 with at least 85% sequence identity to the amino acid sequence of β₂AR is also within the invention. Preferably, the peptide shares 100% sequence identity with the amino acid sequence of β₂AR.

“Identity”, as used herein, refers to the subunit sequence similarity between two polymeric molecules, e.g., two peptide molecules. When a subunit position in both of the two molecules is occupied by the same monomeric subunit, e.g., if a position in each of two peptides is occupied by serine, then they are identical at that position. The identity between two sequences is a direct function of the number of matching or identical positions, e.g., if half (e.g., 5 positions in a polymer 10 subunits in length), of the positions in two peptide or compound sequences are identical, then the two sequences are 50% identical; if 90% of the positions, e.g., 9 of 10, are matched, the two sequences share 90% sequence identity. By way of example, the amino acid sequences VRGLQP and HAFLQP share 50% sequence identity.

Gene Therapy

According to the present invention, a method is provided of supplying β₂AR function to airway cells of an appropriate subject. The wild-type β₂AR gene or a part of the gene may be introduced into the cell in a vector such that the gene remains extrachromosomal. In such a situation, the gene will be expressed by the cell from the extrachromosomal location. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of the routineer.

Gene therapy is carried out according to generally accepted methods. Generally, a virus or plasmid vector, containing a copy of the β₂AR gene linked to expression control elements and capable of replicating inside the airway cells, is prepared. Suitable vectors are known, such as disclosed in U.S. Pat. No. 5,252,479, incorporated herein in its entirety by reference. The vector is then introduced into the patient, either locally at the site of the airway tissue or systemically. If the transfected gene is not permanently incorporated into the genome of the targeted cells, the treatment may have to be repeated periodically.

Gene transfer systems known in the art may be useful in the practice of the gene therapy methods of the present invention. These include viral and nonviral transfer methods. A number of viruses have been used as gene transfer vectors, including papovaviruses, e.g., SV40, adenovirus, vaccinia virus, adeno-associated virus, herpesviruses including HSV and EBV, and retroviruses of avian, murine and human origin. Most human gene therapy protocols have been based on disabled murine retroviruses.

Nonviral gene transfer methods known in the art include chemical techniques such as calcium phosphate co-precipitation; mechanical techniques, for example microinjection; membrane fusion-mediated transfer via liposomes; and direct DNA uptake and receptor-mediated DNA transfer. Viral-mediated gene transfer can be combined with direct in vivo gene transfer using liposome delivery. Alternatively, the retroviral vector producer cell line can be injected into airway tissues. Injection of producer cells would then provide a continuous source of vector particles.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged.

Liposome/DNA complexes have been shown to be capable of mediating direct in vivo gene transfer. While in standard liposome preparations the gene transfer process is nonspecific, localized in vivo uptake and expression have been reported in tumor deposits, for example, following direct in situ administration.

Gene transfer techniques that target DNA directly to lung tissues, e.g., airway smooth muscle and epithelial cells, is preferred. Receptor-mediated gene transfer, for example, is accomplished by the conjugation of DNA (usually in the form of covalently closed supercoiled plasmid) to a protein ligand via polylysine. Ligands are chosen on the basis of the presence of the corresponding ligand receptors on the cell surface of the target cell/tissue type. These ligand-DNA conjugates can be injected directly into the blood if desired and are directed to the target tissue where receptor binding and internalization of the DNA-protein complex occurs. To overcome the problem of intracellular destruction of DNA, co-infection with adenovirus can be included to disrupt endosome function.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). Preferably, when the subject is a human, a vector such as the gibbon ape leukemia virus (GaLV) is utilized. A number of additional retroviral vectors can incorporate multiple genes. All of these vectors can transfer or incorporate a gene for a selectable marker so that transduced cells can be identified and generated. By inserting a β₂AR gene sequence (including promoter region) of interest into the viral vector, along with another gene that encodes the ligand for a receptor on a specific target cell, for example, the vector is now target specific. Retroviral vectors can be made target specific by inserting, for example, a polynucleotide encoding a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the β₂AR polynucleotide.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence that enables the packaging mechanism to recognize an RNA transcript for encapsidation. Helper cell lines that have deletions of the packaging signal include but are not limited to PSI 2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Another targeted delivery system for β₂AR polynucleotide is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes arc artificial membrane vesicles that are useful as delivery vehicles in vitro and in vivo. It has been shown that large unilamellar vesicles (LUV), which range in size from 0.2-4.0 μm can encapsulate a substantial percentage of aqueous buffer containing large macromolecules. RNA, DNA and intact virions can be encapsulated within the aqueous interior and be delivered to cells in a biologically active form. In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

The constructs for use in the invention include several forms, depending upon the intended use of the construct. Thus, the constructs include vector, transcriptioned cassettes, expression cassettes and plasmids. The transcriptional and translational initiation region (also sometimes referred to as a “promoter,”), preferably comprises a transcriptional initiation regulatory region and a translational initiation regulatory region of untranslated 5′ sequences, “ribosome binding sites,” responsible for binding mRNA to ribosomes and translational initiation. It is preferred that all of the transcriptional and translational functional elements of the initiation control region are derived from or obtainable from the same gene. In some embodiments, the promoter will be modified by the addition of sequences, such as enhancers, or deletions of nonessential and/or undesired sequences. By “obtainable” is intended a promoter having a DNA sequence sufficiently similar to that of a native promoter to provide for the desired specificity of transcription of a DNA sequence of interest. It includes natural and synthetic sequences as well as sequences which may be a combination of synthetic and natural sequences.

The nucleic acid constructs generally will be provided as transcriptional cassettes. An intron optionally may be included in the construct, preferably >/=100 bp and placed 5′ to the coding sequence. Generally it is preferred that the construct not become integrated into the host cell genome and the construct is introduced into the host as part of a non-integrating expression cassette. A coding sequence is “operably linked to” or “under the control of” transcriptional regulatory regions in a cell when DNA polymerase will bind the promoter sequence and transcribe the coding sequence into mRNA, either a sense strand or an antisense strand. Thus, the nucleic acid sequence includes DNA sequences that encode polypeptides have the biological activity of β₂AR that are directly or indirectly responsible for a therapeutic effect, as well as nucleotide sequences coding for nucleotide sequences such as antisense sequences and ribozymes.

In one embodiment, constructs are used that produce long-term effects in vivo, either by integration into host cell genomic DNA at high levels or by persistence of the transcription cassette in the nucleus of cells in vivo in stable, episomal form. Integration of the transcription cassette into genomic DNA of host cells in vivo is facilitated by administering the transgene in a linearized form (either in the coding region alone, or the coding region together with 5′ and 3′ regulatory sequences, but without any plasmid sequences present).

The constructs for use in the invention include several forms, depending upon the intended use of the construct. Thus, the constructs include vectors, transcriptional cassettes, expression cassettes and plasmids. The transcriptional and translational initiation region (also sometimes referred to as a “promoter,”), preferably comprise a transcriptional initiation regulatory region and a translational initiation regulatory region of untranslated 5′ sequences, “ribosome binding sites,” responsible for binding mRNA to ribosomes and translational initiation. It is preferred that all of the transcriptional and translational functional elements of the initiation control region are derived from or obtainable from the same gene. In some embodiments, the promoter will be modified by the addition of sequences, such as enhancers, or deletions of nonessential and/or undesired sequences. By “obtainable” is intended a promoter having a DNA sequence sufficiently similar to that of a native promoter to provide for the desired specificity of transcription of a DNA sequence of interest. It includes natural and synthetic sequences as well as sequences which may be a combination of synthetic and natural sequences.

For the transcriptional initiation region, or promoter element, any region may be used with the proviso that it provides the desired level of transcription of the DNA sequence of interest. The transcriptional initiation region may be native to or homologous to the host cell, and/or to the DNA sequence to be transcribed, or foreign or heterologous to the host cell and/or the DNA sequence to be transcribed. By foreign to the host cell is intended that the transcriptional initiation region is not found in the host into which the construct comprising the transcriptional initiation region is to be inserted. By foreign to the DNA sequence is intended a transcriptional initiation region that is not normally associated with the DNA sequence of interest. Efficient promoter elements for transcription initiation include the SV40 (simian virus 40) early promoter, the RSV (Rous sarcoma virus) promoter, the Adenovirus major late promoter, and the human CMV (cytomegalovirus) immediate early 1 promoter.

Inducible promoters also find use with the subject invention where it is desired to control the timing of transcription. Examples of promoters include those obtained from a beta -interferon gene, a heat shock gene, a metallothionein gene or those obtained from steroid hormone-responsive genes, including insect genes such as that encoding the ecdysone receptor. Such inducible promoters can be used to regulate transcription of the transgene by the use of external stimuli such as interferon or glucocorticoids. Since the arrangement of eukaryotic promoter elements is highly flexible, combinations of constitutive and inducible elements also can be used. Tandem arrays of two or more inducible promoter elements may increase the level of induction above baseline levels of transcription which can be achieved when compared to the level of induction above baseline which can be achieved with a single inducible element.

Generally, the regulatory sequence comprises DNA up to about 1.5 Kb 5′ of the transcriptional start of a gene, but can be significantly smaller. This regulatory sequence may be modified at the position corresponding to the first codon of the desired protein by site-directed mutagenesis or by introduction of a convenient linker oligonucleotide by ligation, if a suitable restriction site is found near the N-terminal codon. In the ideal embodiment, a coding sequence with a compatible restriction site may be ligated at the position corresponding to codon #1 of the gene. This substitution may be inserted in such a way that it completely replaces the native coding sequence and thus the substituted sequence is flanked at its 3′ end by the gene terminator and polyadenylation signal.

Transcriptional enhancer elements optionally may be included in the expression cassette. By transcriptional enhancer elements is intended DNA sequences which are primary regulators of transcriptional activity and which can act to increase transcription from a promoter element, and generally do not have to be in the 5′ orientation with respect to the promoter in order to enhance transcriptional activity. The combination of promoter and enhancer element(s) used in a particular expression cassette can be selected by one skilled in the art to maximize specific effects. Different enhancer elements can be used to produce a desired level of transgene expression in a wide variety of tissue and cell types. For example, the human CMV immediate early promoter-enhancer element can be used to produce high-level transgene expression in many different tissues in vivo.

Examples of other enhancer elements that confer a high level of transcription on linked genes in a number of different cell types from many species include enhancers from SV40 and RSV-LTR. The SV40 and RSV-LTR are essentially constitutive. They may be combined with other enhancers that have specific effects, or the specific enhancers may be used alone. Thus, where specific control of transcription is desired, efficient enhancer elements that are active only in a tissue-, developmental-, or cell-specific fashion include immunoglobulin, interleukin-2 (IL-2) and beta-globin enhancers are of interest. Tissue-, developmental-, or cell-specific enhancers can be used to obtain transgene expression in particular cell types, such as B-lymphocytes and T-lymphocytes, as well as myeloid, or erythroid progenitor cells. Alternatively, a tissue-specific promoter such as that derived from the β₂AR gene can be fused to a very active, heterologous enhancer element, such as the SV40 enhancer, in order to confer both a high level of transcription and tissue-specific transgene transcription. In addition, the use of tissue-specific promoters, such as LCK, may allow targeting of transgene transcription to T lymphocytes. Tissue specific transcription of the transgene may be important, particularly in cases where the results of transcription of the transgene in tissues other than the target tissue would be deleterious.

Tandem repeats of two or more enhancer elements or combinations of enhancer elements may significantly increase transgene expression when compared to the use of a single copy of an enhancer element; hence enhancer elements find use in the expression cassette. The use of two different enhancer elements from the same or different sources flanking or within a single promoter can in some cases produce transgene expression in each tissue in which each individual enhancer acting alone would have an effect, thereby increasing the number of tissues in which transcription is obtained. In other cases, the presence of two different enhancer elements results in silencing of the enhancer effects. Evaluation of particular combinations of enhancer elements for a particular desired effect or tissue of expression is within the level of skill in the art.

Although generally it is not necessary to include an intron in the expression cassette, an intron comprising a 5′ splice site (donor site) and a 3′ splice site (acceptor site) separated by a sufficient intervening sequence to produce high level, extended in vivo expression of a transgene administered iv or ip can optionally be included. Generally, an intervening sequence of about 100 bp produces the desired expression pattern and/or level, but the size of the sequence can be varied as need to achieve a desired result. The optional intron placed 5′ to the coding sequence results in high level extended in vivo expression of a transgene administered iv or ip but generally is not necessary to obtain expression. Optimally, the 5′ intron specifically lacks cryptic splice sites that result in aberrantly spliced mRNA sequences.

Alternatively, the intervening sequence may be placed 3′ to the translational stop codon and the transcriptional terminator or inside the coding region. The intron can be a hybrid intron with an intervening sequence or an intron taken from a genomic coding sequence. An intron 3′ to the coding region, a 5′ intron which is of less than 100 bp, or an intron which contains cryptic splice sites may under certain condition substantially reduce the level of transgene expression produced in vivo. However, unexpectedly, a high level of in vivo expression of a transgene can be achieved using a vector that lacks an intron. Such vectors therefore are of particular interest for in vivo transfection.

Downstream from and under control of the transcriptional initiation regulatory regions is a multiple cloning site for insertion of a nucleic acid sequence of interest that will provide for one or more alterations of host genotype and modulation of host phenotype. Conveniently, the multiple cloning site may be employed for a variety of nucleic acid sequences in an efficient manner. The nucleic acid sequence inserted in the cloning site may have any open reading frame encoding a polypeptide of interest, for example, an enzyme, with the proviso that where the coding sequence encodes a polypeptide of interest, it should lack cryptic splice sites which can block production of appropriate mRNA molecules and/or produce aberrantly spliced or abnormal mRNA molecule. The nucleic acid sequence may be DNA; it also may be a sequence complementary to a genomic sequence, where the genomic sequence may be one or more of an open reading frame, an intron, a non-coding leader sequence, or any other sequence where the complementary sequence will inhibit transcription, messenger RNA processing, for example splicing, or translation.

The incidence of integration of the transcription cassette into genomic DNA may be increased by incorporating a purified retroviral enzyme, such as the HIV-1 integrase enzyme, into the lipid carrier-DNA complex. Appropriate flanking sequences are placed at the 5′ and 3′ ends of the nucleic acid. These flanking sequences have been shown to mediate integration of the HIV-1 DNA into host cell genomic DNA in the presence of HIV-1 integrase. Alternatively, the duration of the expression of the exogenous nucleic acid in vivo can be prolonged by the use of constructs that contain non-transforming sequences of a virus such as Epstein-Barr virus, and sequences such as oriP and EBNA-1 which appear to be sufficient to allow heterologous DNA to be replicated as an episome in mammalian cells.

The termination region employed primarily will be one of convenience, since termination regions appear to be relatively interchangeable. The termination region may be native to the intended nucleic acid sequence of interest, or may be derived from another source. Convenient termination regions are available and include the 3′ end of a gene terminator and polyadenylation signal from the same gene from which the 5′ regulatory region is obtained. Adenylation residues, preferably more than 32 and up to 200 or more as necessary may be included in order to stabilize the mRNA. Alternatively, a terminator and polyadenylation signal from different gene/genes may be employed with similar results. Specific sequences which regulate post-transcriptional mRNA stability may optionally be included. For example, certain polyA sequences and beta-globin mRNA elements can increase mRNA stability, whereas certain AU-rich sequences in mRNA can decrease mRNA stability. In addition, AU regions in 3′ non-coding regions may be used to destabilize mRNA if a short half-life mRNA is desirable for the gene of interest.

Aerosol Administration

The mammalian host may be any mammal. Thus, the subject application finds use in domestic animals, e.g., equine, feed stock, such as bovine, ovine, and porcine, as well as primates, particularly humans. In the method of the invention, transformation in vivo is obtained by introducing a non-integrating therapeutic plasmid into the mammalian host, preferably complexed to a lipid carrier, particularly a cationic lipid carrier more particularly, for human use or for repeated applications a biodegradable lipid carrier. For introduction into the mammalian host any physiologically acceptable medium may be employed for administering the DNA or lipid carriers, such as deionized water, 5% dextrose in water, and the like. Other components may be included in the formulation such as stabilizers, biocides, etc, providing that they meet the criteria outlined above, i.e. do not cause aggregation of the complexes. The various components listed above find extensive exemplification in the literature and need not be described in particular here.

For aerosol delivery in humans or other primates, the aerosol is generated by a medical nebulizer system that delivers the aerosol through a mouthpiece, facemask, etc. from which the mammalian host can draw the aerosol into the lungs. Various nebulizers are known in the art and can be used in the method of the present invention. The selection of a nebulizer system depends on whether alveolar or airway delivery (i.e., trachea, primary, secondary or tertiary bronchi, etc.), is desired. The particular nucleic acid composition is chosen that is not too irritating at the required dosage.

Nebulizers useful for airway delivery include those typically used in the treatment of asthma. Such nebulizers are also commercially available. The amount of compound used will be an amount sufficient to provide for adequate transfection of cells after entry of the DNA or complexes into the lung and airway and to provide for a therapeutic level of transcription and/or translation in transfected cells. A therapeutic level of transcription and/or translation is a sufficient amount to prevent, treat, or palliate a disease of the host mammal following administration of the nucleic acid composition to the host mammal's lung, particularly the bronchopulmonary and bronchiolopulmonary smooth muscle and epithelial cells of the trachea, bronchi, bronchia, bronchioli, and alveoli. Thus, an effective amount of the aerosolized nucleic acid preparation, is a dose sufficient to effect treatment, that is, to cause alleviation or reduction of symptoms, to inhibit the worsening of symptoms, to prevent the onset of symptoms, and the like. The dosages of the preset compositions that constitute an effective amount can be determined in view of this disclosure by one of ordinary skill in the art by running routine trials with appropriate controls. Comparison of the appropriate treatment groups to the controls will indicate whether a particular dosage is effective in preventing or reducing particular symptoms.

The total amount of nucleic acid delivered to a mammalian host will depend upon many factors, including the total amount aerosolized, the type of nebulizer, the particle size, breathing patterns of the mammalian host, severity of lung disease, concentration of the nucleic acid composition in the aerosolized solution, and length of inhalation therapy. Thus, the amount of expressed protein measured in the airways may be substantially less than what would be expected to be expressed from the amount of nucleic acid present in the aerosol, since a large portion of the complex may be exhaled by the subject or trapped on the interior surfaces of the nebulizer apparatus. For example, approximately one third of the nucleic acid composition dose that is placed into the nebulizer remains in the nebulizer and associated tubing after inhalation is completed. This is true regardless of the dose size, duration of inhalation, and type of nebulizer used. Moreover, resuspension of the residue and readministration does not significantly increase the dose delivered to the subject; about one third remains in the nebulizer. Additionally, efficiency of expression of the encoded protein will vary widely with the expression system used.

Despite the interacting factors described above, one of ordinary skill in the art will be able readily to design effective protocols, particularly if the particle size of the aerosol is optimized. Based on estimates of nebulizer efficiency, an effective dose delivered usually lies in the range of about 1 mg/treatment to about 500 mg/treatment, although more or less may be found to be effective depending on the subject and desired result. It is generally desirable to administer higher doses when treating more severe conditions. Generally, the nucleic acid is not integrated into the host cell genome, thus if necessary, the treatment can be repeated on an ad hoc basis depending upon the results achieved. If the treatment is repeated, the mammalian host is monitored to ensure that there is no adverse immune response to the treatment. The frequency of treatments depends upon a number of factors, such as the amount of nucleic acid composition administered per dose, as well as the health and history of the subject.

Experimental Procedures

Transgenic Mice. Smooth muscle specific expression of the human β₂AR in transgenic mice was achieved by using the mouse smooth muscle α-actin promoter (18) (a gift from Dr. A. Strauch). The construct was prepared by cloning the 1.5 kb Hind III/PshA I fragment encoding the human β₂AR (1.2 kb of ORF and 0.3 kb of 3′ UTR) upstream of the SV40 polyadenylation sequence in the plasmid pNNO3. The 3.6 kb smooth muscle α-actin promoter fragment, termed SMP8 (18), was then subcloned into a BamH I site 5′ to the β₂AR ORF. Orientation of each fragment was confirmed by sequence analysis and restriction enzyme digestion. The transgenic construct (˜5.9 kb) was excised from the plasmid by Not I digestion and microinjected into male pronuclei of fertilized zygotes from superovulated FVB/N mice. Surviving zygotes were implanted into pseudopregnant foster mothers who gave birth to founders. Transgene positive founder mice were identified by Southern blot analysis of genomic DNA derived from tail clips. Founders expressing the transgene were mated with nontransgenic FVB/N mice. Subsequent screening for the hemizygous transgene positive progeny was by PCR analysis of the genomic DNA using a forward primer in the β₂AR ORF (5′-GGAGCAGAGTGGATATCACG-3′) (SEQ ID NO:3) and a reverse primer in the SV40 polyadenylation region (5′-GTCACACCACAGAAGTAAGG-3′) (SEQ ID NO:4). Hemizygous mice from generations 2-4 between the ages of 10 and 14 weeks were used for all studies.

RNA Studies. Ribonuclease protection assays (RPA) were used to quantitate the amount of transgene mRNA in total RNA prepared from whole lung homogenates. A [³²P]-labeled antisense riboprobe corresponding to the distal 500 bp of the human β₂AR ORF was prepared. This portion of the human transcript has only ˜75% homology with the mouse β₂AR mRNA. A radiolabeled antisense riboprobe for β-actin was also utilized to account for any potential differences in gel loading. RPAs were carried out as previously reported (19) by hybridizing 20 μg of total cellular RNA with both the β₂AR and actin riboprobes. To localize transgene expression within the lung in situ hybridization was performed on lung sections as previously described (20). Briefly, lungs were rapidly dissected, fixed in 4% paraformaldehyde, cryoprotected with 30% sucrose in PBS, and frozen in OCT. Cryostat sections (7 μm) were then mounted on saline-coated slides. An antisense cRNA probe for the human β₂AR was prepared as described above for the RPA studies except that the probe was labeled with [³⁵S]UTP using a commercially available kit (Stratagene). A sense cRNA probe was generated from the same plasmid template using SP6 polymerase for use as a negative control. Hybridization was performed with 0.5-1.0×10⁶ cpm of labeled probe in a final volume of 30 μl per slide. Following overnight incubation at 42° C., the sections were treated with 50 μg/ml RNase A and 100 U/ml RNAse T1 for 30 min at 37° C., and washed to a final stringency of 0.1× standard citrate saline at 50° C. Slides were dipped in NTB2 emulsion (Eastman Kodak Co.) diluted 1:1 with 0.6 M ammonium acetate, exposed for 2 weeks, after which they were developed with D19 developer (Eastman Kodak Co.) and counterstained with hematoxylin and cosin.

Airway Smooth Muscle Cell Cultures. Tracheal smooth muscle cells were cultured from explants of excised tracheas using a modification of previously described methods (21). The entire trachea between the larynx and mainstem bronchi was removed and placed in a sterile petri dish containing room temperature HBSS supplemented with a 2× concentration of antibiotic-antimycotic solution (Gibco BRL). After additional surrounding tissue was removed with the aid of a dissecting microscope, the tracheal segment was split longitudinally and dissected into 2-3 mm squares. All the segments from a single trachea were then placed intima side down in a sterile 60 mm dish. After allowing the explants to adhere, 2.5 ml of DMEM supplemented with 20% FCS and 2× antibiotic-antimycotic was added to cover the explants. The explants were incubated at 37° C. in a humidified environment of 95% air-5% CO₂. After the first 3 days of cell growth, the concentration of FCS was reduced to 10% and the antibiotic-antimycotic to 1×. Explanted trachea was removed when the outgrowing cells became locally confluent. Once the 60 mm dish became confluent, the cells were harvested by trypsinization and passed into a single 75 cm² flask. Tracheal smooth muscle cells were subsequently passaged at a 1:4 ratio. Greater than 90% of these cells from each donor mouse were smooth muscle cells, as determined by immunohistochemistry performed with an antibody raised against smooth muscle α-actin (see below). All experiments were performed on confluent cells at matched passage numbers 3 to 6.

Radioligand Binding and cAMP Studies. To prepare membranes from lung tissue, lungs from an individual mouse were homogenized with a Polytron (Brinkman) in 10 ml of hypotonic lysis buffer (5 mM Tris, pH 7.4, 2 mM EDTA) containing the protease inhibitors leupeptin, aprotinin, benzamidine and soybean trypsin inhibitor (10 μg/ml each). Detached smooth muscle cells from primary cultures were processed similarly. Homogenates were centrifuged at 40,000×g for 10 min at 4° C. The pellets were washed and centrifuged two additional times, after which the pellets were suspended in assay buffer (75 mM Tris, pH 7.4, 12.5 MM MgCl₂, 2 mM EDTA). For determination of receptor expression, radioligand binding was carried out with [¹²⁵I]CYP as described (22). The fraction of receptors in the high-affinity binding state was determined by competition experiments performed in the absence of GTP using 40 pM [¹²⁵I]CYP and 16 concentrations of isoproterenol ranging from 10⁻⁴ M to 10⁻¹⁰ M as previously described (23). Competition data were fit to a two-site model when this fit was statistically (p<0.05) better than a one-site fit by F-test, using Prizm software (GraphPad, San Diego Calif.). Receptor density on the cell surface was assessed using methods previously described (24). Briefly, cells grown in monolayers were detached with 0.25% trypsin for 5 min at 37° C. After trypsin activity was neutralized by the addition of FCS, the cells were washed with PBS and resuspended in DMEM. The resuspended cells were incubated in a volume of 500 μl with 400 pM [¹²⁵I]CYP at 37° C. for 60 min in the absence or presence of the hydrophobic antagonist propranolol (1 μM) or the hydrophilic antagonist CGP 12177 (10 μM). Binding to receptors localized throughout the cell was defined as that displaced by propranolol, while cell surface binding was defined as that displaced by CGP12177 (24). Bound radioactivity was separated by filtration and washing over GF/C glass fiber filters. Cyclic AMP content of attached mouse smooth muscle cells in culture exposed to 10 82 M isoproterenol or carrier for 10 min was measured by an acetylated radioimmunoassay method as described (19). Adenylyl cyclase activity was measured in membranes prepared as above using column chromatography as previously described (25).

Ex vivo Smooth Muscle Studies. Studies of mouse tracheal contractility have been reported in detail elsewhere (26). Briefly, tracheas were excised and dissected free of surrounding tissues and cut into rings of approximately 5 mm in length. The trachea rings were mounted on stainless steel wires connected to isometric force transducers. The rings were then immersed in a physiologic saline solution (in mM: NaCl 118, KCl 4.73, MgCl₂ 1.2, EDTA 0.026, KH₂PO₄ 1.2, CaCl₂ 2.5, NaH₂CO₃ 25 and glucose 11) maintained at 37° C. and bubbled with 95% O₂/5% CO₂ to maintain a pH of 7.4. Each tracheal ring was stretched to a tension of 5 mN, an optimal passive tension for maximizing active force (27). After a 20 min equilibration period, contraction/relaxation cycles with 10 μM acetylcholine were performed until consistent forces were observed. Cumulative concentration-isometric force curves were then generated to acetylcholine (1 nM-30 μM). After rinsing, tracheas were contracted with 10 μM acetylcholine (approximately the ED₈₀) and cumulative concentration-relaxation curves were generated to isoproterenol (30 pM-10 μM). Concentration-response relations were fitted to a logistic equation (26).

In Vivo Airway Physiology. Airway responsiveness to methacholine was measured noninvasively in conscious, unrestrained mice using a whole body plethysmograph (Buxco Electronics, Troy N.Y.) (28). Using this system, the volume changes that occur during a normal respiratory cycle are recorded as the pressure difference between the animal-containing chamber and a reference chamber. The resulting signal is used to calculate respiratory frequency, minute volume, tidal volume, and enhanced pause (Penh). Penh is a unitless value that is a function of the peak inspiratory (PIP) and peak expiratory (PEP) pressures and the timing of expiration and is calculated as:

Penh=Pause×(PEP/PIP)

where Pause is the ratio of time spent in the last third of expiration relative to early expiration. Penh has been shown to closely correlate with invasive measurements of airway resistance (28) and was used as the measure of airway responsiveness in this study. Mice were placed in the chamber and allowed to adjust to their surroundings for 10 min. They were then exposed to aerosolized PBS (to establish baseline), followed by increasing concentrations of methacholine (2.5 to 80 mg/ml). Aerosolization was for 3 min and respiratory measurements were recorded and averaged for the subsequent 5 min after each dose. The degree of bronchoconstriction was expressed as the percent change in Penh relative to the PBS baseline. On a separate day, the mice were submitted to the same protocol except that they were first treated with aerosolized albuterol (0.1 mg/ml) for 20 min. The concentration-response data for each individual mouse were fit to a sigmoid curve by an iterative least squares technique, and the dose of methacholine required to double baseline Penh (ED₂₀₀) was derived.

Results and Discussion

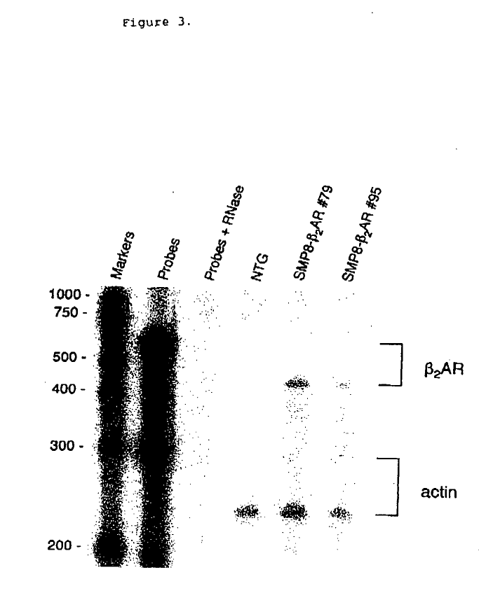

From a total of 40 mice screened, three SMP8-β₂AR founder mice were identified. Subsequent matings with nontransgenic mice showed that one of the three founders was mosaic. In lines established from the other two founder mice (denoted as lines 79 and 95), the transgene was inherited in ˜50% of the offspring with equal distribution between male and female mice. Hemizygous mice from these two lines were further characterized with respect to transgene copy number, mRNA expression, receptor density, and histologic analysis. Southern blot analysis of genomic DNA prepared from tail clips showed that the transgene copy number was ˜1 copy for line 95 and ˜2 copies for line 79 (data not shown). To confirm that the SMP8-β₂AR transgene was being expressed in the lungs of the transgenic positive mice, total cellular RNA prepared from whole lung homogenates of transgenic and nontransgenic animals was subjected to ribonuclease protection assays. For these experiments, we used an antisense RNA probe corresponding to the distal 500 bp of the human β₂AR ORF. Because this region has <75% homology with the mouse sequence, only the human sequence results in a full-length protected fragment. As shown in FIG. 3, SMP8-β₂AR mRNA was present in lungs from mice that screened positive for the transgene, but was absent in nontransgenic littermates. Quantitation of band density showed that there was no significant difference in transgene expression between lines 79 and 95.

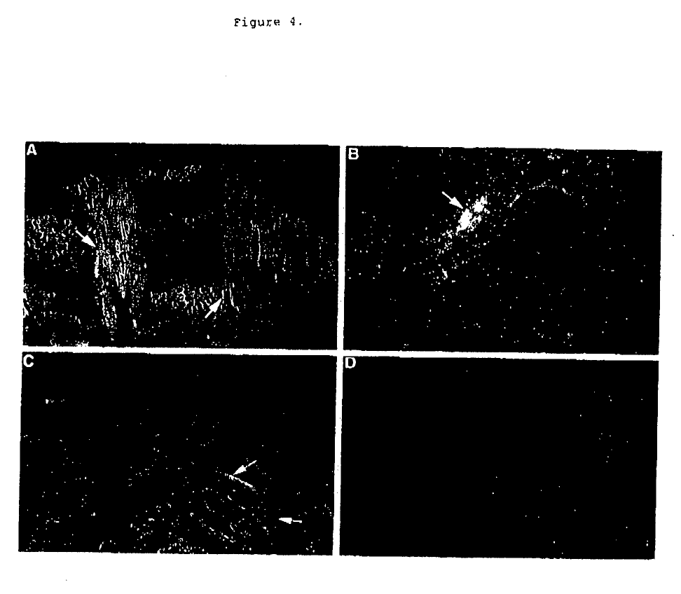

In situ hybridization was performed to verify that expression of the transgene was directed to airway smooth muscle. Previous studies have shown that β₂AR mRNA is heavily expressed in airway smooth muscle (5,7). We therefore used the same species-specific cRNA probe described above for the RPA analysis so that we could limit detection to that of the transgene only. As shown in FIG. 4, specific hybridization (appearing as white dots) was observed in the SMP8-β₂AR mice (Panels A and B), but was absent in nontransgenic mice (Panels C and D). These studies clearly show that expression of the SMP8-β₂AR transcript was confined to airway smooth muscle, and to a lesser extent in pulmonary vascular smooth muscle, with no signal observed in the bronchial epithelium or alveolar lining cells (FIG. 4, Panel A and B). Additional studies with in situ hybridization or radioligand binding showed increased expression in smooth muscle of stomach, colon, and uterus (data not shown).

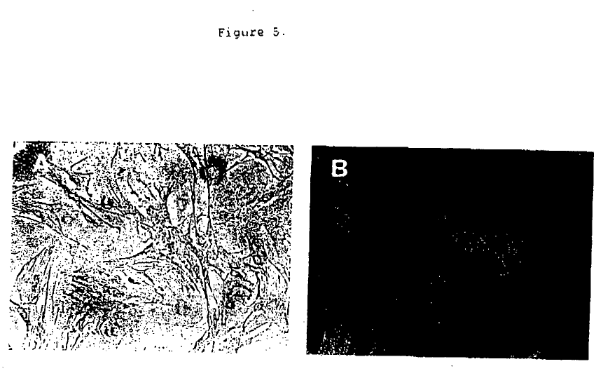

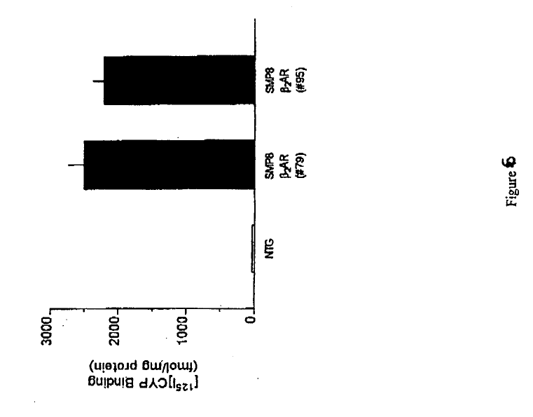

β₂AR expression was further quantitated by radioligand binding assays with [¹²⁵I]CYP. Initial studies with membrane preparations from whole lung homogenates showed no differences in receptor density between transgenic and nontransgenic mice. However, previous studies have shown that >90% of β₂AR's in the lung are localized to cells (type I and type II pneumocytes and capillary endothelium) that line the alveoli (5,7). Indeed, based on the densities of βAR as assessed by autoradiography (5,7) and the extensive surface area of alveoli (29), the ratio of total airway smooth muscle vs alveolar βAR is ˜1:1000 or more. Thus we felt it was unlikely that using a whole lung preparation would detect enhanced smooth muscle expression in the transgenic mice. Since the in situ hybridization experiments demonstrated transgene expression in airway smooth muscle, we measured [¹²⁵I]CYP binding in smooth muscle cells cultured from tracheal explants. As indicated in FIG. 5, immunohistochemistry studies showed that >90% of these cells had the morphologic characteristics of smooth muscle cells and stained positive for smooth muscle α-actin. Saturation radioligand binding studies showed that the β₂AR was significantly overexpressed in tracheal smooth muscle cells from transgenic mice when compared to the level of expression in smooth muscle cells derived from nontransgenic mice (FIG. 6). βAR density in airway smooth muscle cells from transgenic lines 79 and 95 were both 75 times greater than that of cells from nontransgenic mice (2510±229 and 2218±167 vs 33±6 fmol/mg protein, respectively, n=4, p<0.001). Given that the levels of transgene mRNA and receptor protein were equivalent in the two transgenic lines, the majority of the remaining pharmacological and physiological studies were carried out with mice of line 95.

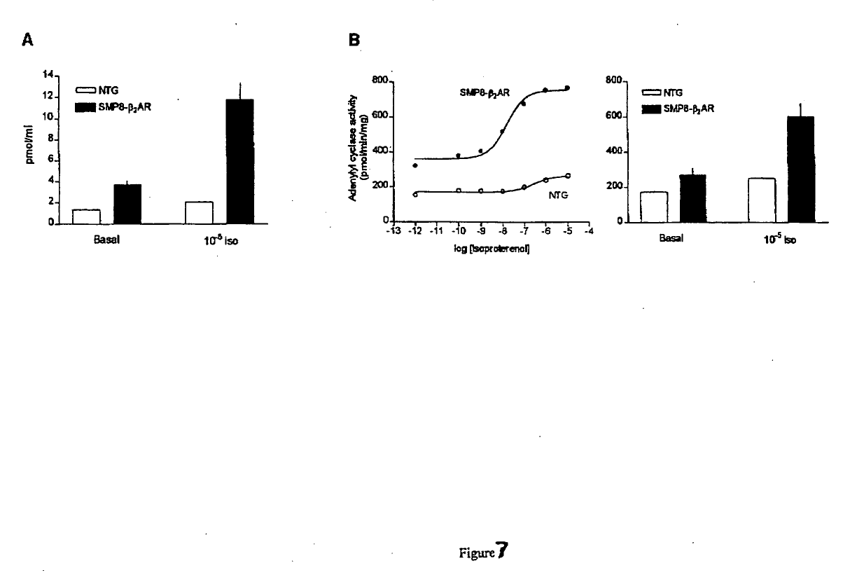

Radioligand binding with [¹²⁵I]CYP was also carried out in whole cells, using the competitors propranolol (hydrophobic) and CGP12177 (hydrophilic) to identify total cellular vs cell surface β₂AR, respectively. These studies showed that the vast majority of β₂AR (95±1.5%, n=4) of transgenic smooth muscle cells were expressed on the cell surface. This distribution was similar to what was found in cells from nontransgenic mice, although quantitation was difficult due to the low expression of receptors in these cells. Having determined that the transgenic β₂AR have a normal cellular distribution, we next assessed whether they had the capacity to form the high-affinity agonist/receptor/Gₛ complex. In cells derived from nontransgenic and transgenic mice, agonist (isoproterenol) competition data in studies carried out in the absence of guanine nucleotide were best fit to a two-site model, while in the presence of 100 μM GTP the data were best fit to a single site model. However, in the absence of GTP the proportion of receptors in the high affinity state (% R_{H}) was lower in membranes from transgenic cells compared to those of nontransgenic cells (% R_{H}=18±4.2 vs 36±6.7). Taken together, the radioligand binding studies indicate that expression of β₂AR is significantly greater in transgenic smooth muscle cells compared to nontransgenic, that such expression is localized primarily to the cell surface, but that a smaller fraction of these receptors is capable of physically coupling to Gₛ. To assess whether receptor-mediated adenylyl cyclase stimulation was enhanced in SMP8-β₂AR mice, we measured both cAMP content (FIG. 7a) in intact tracheal smooth muscle cells and adenylyl cyclase activities in smooth muscle cell membranes (FIG. 7b). We found that basal cAMP contents in airway smooth muscle cells from transgenic mice were ˜2.8-fold greater than those of cells from nontransgenic mice (3.70±0.41 vs 1.34±0.05 pmol/ml, respectively, n=5, p<0.001). Similarly, isoproterenol-stimulated cAMP content in transgenic airway smooth muscle cells was also significantly greater than that of cells from the nontransgenic controls (11.78±1.62 vs 2.05±0.10 pmol/ml, respectively, n=5 p<0.001). When assessed as percent-stimulation over basal levels, the isoproterenol-stimulated cAMP levels of cells from the transgenic mice (˜200%) was markedly greater than what was observed in nontransgenic cells, which amounted to only ˜50% over basal. Studies of adenylyl cyclase activity in cell membranes gave similar results. Basal activities were greater in transgenic as compared to nontransgenic membranes (273±39.3 vs 172±7.8, n=4, p<0.05), as were maximal isoproterenol stimulated activities (609±74.7 vs. 251±7.3, n=4, p<0.005). The percent isoproterenol stimulation over basal was ˜125% vs ˜46%, respectively. The dose-response curve for isoproterenol stimulation of adenylyl cyclase from SMP8-β₂AR membranes was left shifted compared to than doubling of basal adenylyl cyclase or cAMP levels by isoproterenol in cultured airway smooth muscle cells from other species has been previously reported (30), although higher levels have also been noted (21). This variability is likely due to assay conditions or species variation. However, in our work smooth muscle cells from the two lines were studied under identical conditions in a paired manner, so it is the difference observed between transgenic and nontransgenic cells that is the critical finding.

These studies thus clearly demonstrate that overexpression of β₂AR in airway smooth muscle results in enhanced adenylyl cyclase activity at baseline and in response to agonist. The data are consistent with the multistate model of G protein coupled receptors, where in the non-agonist bound state a small proportion of receptors spontaneously achieve the active conformation (R*). With overexpression, the number of receptors in this state at any one time is increased sufficiently to alter “basal” coupling, as shown by the increased cAMP levels and adenylyl cyclase activities of airway smooth muscle cells in the absence of agonist. Maximal constitutive activation of Gₛ-adenylyl cyclase was not observed, though, since isoproterenol resulted in yet further stimulation of cAMP in the transgenic derived cells. The percent stimulation by agonist over baseline was significantly greater as compared to non-transgenic littermates. Taken together, the data are consistent with the β₂AR being a limiting factor in the receptor-Gₛ-adenylyl cyclase cascade in native airway smooth muscle cells. This is in direct contrast to other studies (15-17) which have concluded that there is a substantial receptor reserve on airway smooth muscle. Although β₂AR expression was increased ˜75-fold in the current study, we did not observe commensurate increases in basal or agonist-stimulated cAMP responses. A very similar finding has been reported with transgenic β₂AR overexpression in the heart. In one such study, we obtained a ˜45-fold overexpression of wild-type human β₂AR in the hearts of transgenic mice, yet basal and isoproterenol-stimulated adenylyl cyclase activities were increased only 3-4 fold over nontransgenic activities (31). Milano et al (32) had a 200-fold increase in cardiac β₂AR expression, with 2-fold increases in basal and isoproterenol stimulated activities. These findings have been interpreted as being consistent with other elements of the transduction cascade (Gₛ, adenylyl cyclase) becoming limiting factors when βAR expression is markedly increased, such that proportional increases in signalling in relation to receptor expression is not observed. Indeed, in the current study a smaller percentage of β₂AR in transgenic smooth muscle cells can form the high-affinity receptor-Gₛ, complex as compared to nontransgenic cells, likely indicating insufficient Gₛ to accommodate all the overexpressed receptors.

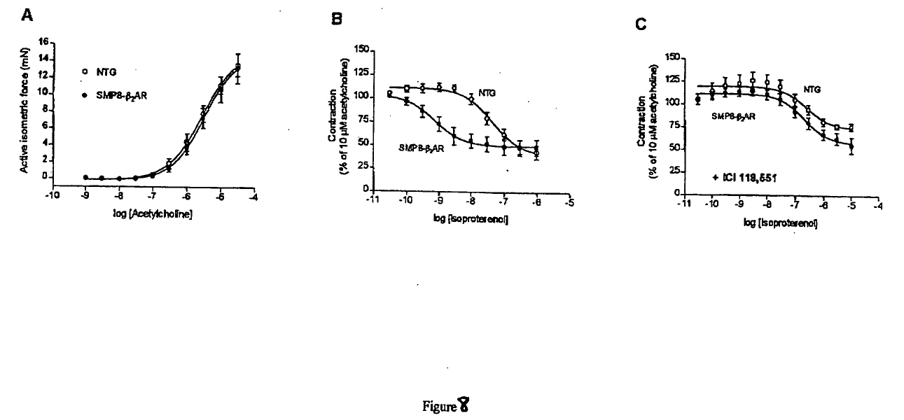

To determine whether this enhanced signalling observed in cells results in regulation of coordinated smooth muscle function of the airway, we measured responses to agonist ex vivo using tracheal ring preparations. For these studies, tracheal rings dissected from SMP8-β₂AR and nontransgenic mice were mounted in the same organ bath and relaxation in response to isoproterenol was measured. Initial studies showed that the constriction response to acetylcholine was equivalent between rings derived from transgenic and nontransgenic mice (FIG. 6a). For isoproterenol dose-response experiments, rings were pre-constricted by incubation with 10 μM acetylcholine. Tile sensitivity to isoproterenol was found to be markedly enhanced in the tracheal rings from SMP8-β₂AR mice (FIG. 8B). As shown, response curves for isoproterenol for these mice were shifted to the left (60 fold decrease in ED₅₀) compared to nontransgenic tracheal rings (ED₅=0.64±0.07 vs 40.0±7.1 nM, respectively, n=4 p<0.001). However, the maximal extent of relaxation was equivalent. This enhancement of isoproterenol-induced relaxation in SMP8-β₂AR mice was blocked by pretreatment with the selective β₂AR antagonist ICI 118,551 (0.1 μM) (FIG. 8C), indicating that the response observed in these mice was directly the result of δ₂AR activation from transgenic overexpression of the receptor rather than a change in some other factor caused by insertion of the transgene. While these differences in smooth muscle relaxation ex vivo are quite significant, the fact that the maximal degree of agonist-mediated relaxation in the transgenic rings was not greater than that of the nontransgenics suggests that at the level of this physiologic response there may be other factors which limit further relaxation regardless of the number of β₂AR expressed. Since in intact smooth muscle cells we do observe a leftward shift in the response curve as well as an increase in the maximal response, the limiting factor(s) may be in elements that are necessary after the early signal transduction events.

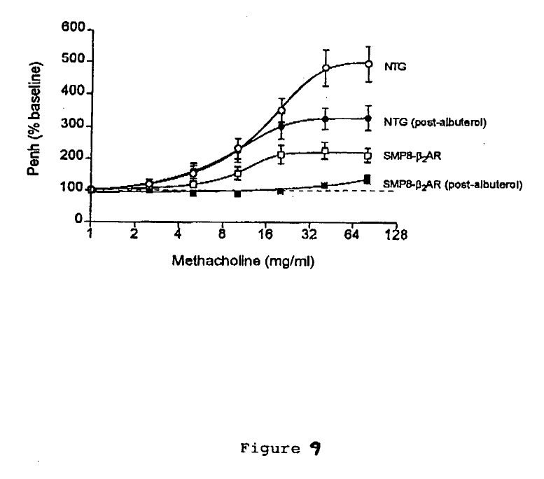

The above results indicated that transgenic mice overexpressing the β₂AR in airway smooth muscle have enhanced signalling in isolated cells and tracheal rings. To determine whether this resulted in altered physiologic function of the airways in vivo, studies were carried out in intact mice using a rodent whole body plethysmography system. We hypothesized that this persistent β₂AR signalling would result in a state of relative hyporesponsiveness to bronchoconstriction by methacholine. Thus, the responses to inhaled methacholine alone, and methacholine after inhalation of the β-agonist albuterol, were measured (FIG. 9). These results show that the maximal level of bronchoconstriction induced by methacholine in the SMP-β₂AR mice was significantly less than that of nontransgenic mice (Penh=250±26% vs 558±42% of baseline, respectively, p<0.001). In addition, the sensitivity to methacholine was altered in the SMP-β₂AR mice, with the ED₂₀₀=35.7±10.6 as compared to 12.1±1.90 mg/ml in the nontransgenic species (p<0.02). Even more striking were the responses to methacholine after exposure to the β-agonist albuterol. As shown, when SMP8-β₂AR mice were pretreated with albuterol, methacholine caused no increase in Penh, even at the highest dose used in this study (80 mg/ml) (FIG. 9). In contrast, in nontransgenic mice methacholine responsiveness after albuterol was present, with the maximal bronchoconstriction being 373±33% of baseline. It is interesting to note that the extent of bronchoconstriction in nontransgenic mice treated with albuterol was greater than that observed in the transgenic mice in the absence of agonist (Penh=373±33% vs 250±26% of baseline, p=0.01). These results are entirely consistent with the intact smooth muscle cell studies, where maximal cAMP content after agonist exposure in the nontransgenic cells was similar to non-agonist exposed levels in the transgenic cells. The in vivo studies are also consistent with the ex vivo organ bath results. Here, trachea overexpressing β₂AR maximally relaxed in the presence of low concentrations of agonist, while nontransgenic rings showed no demonstrable response at these concentrations (FIG. 8b). It should be noted, though, that while cellular cAMP/adenylyl cyclase, tracheal ring, and whole body plethysmography studies are complementary, each has constraints which limit interpretation in isolation. In cells, our studies are confined to measurement of effector (adenylyl cyclase) activity or its product (cAMP). While these experiments, and radioligand binding studies, can assess coupling of the receptor to this pathway, they do not provide information regarding post-cAMP events relevant to smooth muscle function (i.e., relaxation). The tracheal ring studies require pre-constriction with acetylcholine in order to derive a signal, and may not directly relate relaxation to airflow in vivo, the latter being influenced by additional physiologic variables. The whole animal plethysmography studies are limited by the doses of the drugs that are tolerated, could additionally be influenced by endogenous catecholamines, and are not easily amenable to measurements in response to multiple doses of agonist. Given the whole cell cAMP and the in vivo plethysmography results, it would not be unexpected for the tracheal ring studies to show a decreased basal force (tension) and a leftward shift in the acetylcholine dose-response for the transgenic rings. However, tracheal rings must be stretched to some extent in order to obtain a transducer signal. Thus, basal as well as acetylcholine-induced constriction are under conditions in such preparations that are not analogous to our whole cell or in vivo studies.

Our studies constitute the first transgenic overexpression of β₂AR in a tissue where the receptor acts to inhibit a physiologic response. Previous reports with transgenic overexpression of the receptor in the heart, where the receptor stimulates contraction, have shown increased cardiac inotropy and chronotropy (31,32). These studies suggested that β₂AR overexpression might be useful therapeutically to overcome the depressed chronotropic state in congestive heart failure. Subsequent studies with very high β₂AR overexpression in genetic models of cardiac hypertrophy/heart failure have revealed an increased mortality (33,34). In our current work we have overexpressed the receptor in a cell type where the physiologic response of β₂AR is a decrease, rather than an increase, in contraction. In mice up to 14 months old, we have not found structural remodeling of the airway, alterations in smooth muscle morphology, or pathologic consequences in the lung of β₂AR overexpression in airway smooth muscle. Our results clearly indicate that the β₂AR mediated relaxation response in airway smooth muscle can accommodate increased receptor levels with an increase in baseline and agonist-promoted function. The notion that there are sufficient spare receptors on native airway smooth muscle to limit the effectiveness of enhanced expression or function is thus not supported, at least in mouse lung.

The potential for transgenic overexpression of β₂AR in smooth muscle to decrease cellular levels of Gₛ has been suggested in studies with transfection of β₂AR into NG108 cells (35). Such a decrement might have consequences for β₂AR and other Gₛ coupled receptors endogenously expressed on the airway, and could limit the effectiveness of the transgene. Using the isolated airway smooth muscle cells, G_{αs} content as assessed by Western blots was not different in cells derived from transgenic mice compared to nontransgenic littermates (data not shown). Nor were the levels of the Gᵢ isoform G_{αi2} different. We also examined the potential for agonist-promoted desensitization of physiologic β₂AR responses in this setting of transgenic overexpression of the receptor. Transgenic mice were implanted with osmotic minipumps administering isoproterenol for three continuous days, then studied by plethysmography. The methacholine concentration-response curve in transgenic mice pretreated with isoproterenol remained essentially flat after acute albuterol, with a maximal Penh of 128±1% of baseline (compared to 146±12% in the absence of isoproterenol pretreatment, p>0.05). So agonist-promoted desensitization of β₂AR function at the physiologic level is not observed in the SMP8-β₂AR transgenic mice, likely due to the extensive overexpression to a point such that some fraction of spare receptors are in fact present.

In conclusion, we have shown that the β₂AR of airway smooth muscle represent a limiting element of the signal transduction pathway. This constraint is alleviated by increased expression, which enhances basal and isoproterenol stimulated levels of intracellular cAMP. Such an increase has a significant impact on airway smooth muscle function, ultimately decreasing bronchial hyperresponsiveness. Given the central importance of bronchial hyperresponsiveness to the asthmatic phenotype, these mice can be considered in an anti-asthmatic state. As such, overexpression of β₂AR in airway smooth muscle may be a potential genetic therapy for asthma.

Footnotes ¹ The abbreviations are: βAR, β-adrenergic receptor; PKA, protein kinase A; Gₛ, stimulatory guanine nucleotide binding protein; Penh, enhanced pause; NTG, nontransgenic littermates.

### References

1. Green, S. A. and Liggett, S. B. (1996) in The Genetics of Asthma (Liggett, S. and Meyers, D., eds) pp. 67-90, Marcel Dekker, Inc., New York

2. Postma, D. S. and Kerstjens, H. A. (1998) Am J Respir Crit Care Med 158, S187-S192

3. Paul, R. J. and de Lanerolle, P. (1996) in Genetics of Asthma (Liggett, S. and Meyers, D., eds) pp. 91-117, Marcel Dekker, Inc., New York

4. Hakonarson, H. and Grunstein, M. (1998) Am J Respir Crit Care Med 158, S115-S122

5. Carstairs, J. R., Nimmo, A. J., and Barnes, P. J. (1985) Am.Rev.Resp.Dis. 132, 541-547

6. Henry, P. J. and Goldie, R. G. (1990) Br J Pharmacol 99, 131-135

7. Henry, P. J., Rigby, P. J., and Goldie, R. G. (1990) Br J Pharmacol 99, 136-144

8. O'Donnell, S. R. (1972) Eur J Pharmacol 19, 371-379

9. Toda, N., Hayashi, S., Hatano, Y., Okunishi, H., and Miyazaki, M. (1978) J Pharmacol Exp Ther 207, 311-319

10. Barnes, P. J., Cuss, F. M., and Palmer, J. B. (1985) Br J Pharmacol 86, 685-691

11. Liggett, S. B. (1997) in The Lung: Scientific Foundations (Crystal, R., West, J. B., Weibel, E. R., and Barnes, P. J., eds) pp. 19-36, Raven Press, New York

12. Bai, T. R. (1995) Lung 170, 125-141

13. Kenakin, T. (1988) Life Sci 43, 1095-1101

14. Daaka, Y., Luttrell, L. M., and Lefkowitz, R. J. (1997) Nature 390, 88-91

15. Avner, B. P. and Wilson, S. (1979) Proc West Pharmacol Soc 22, 177-181

16. Lemoine, H. and Overlack, C. (1992) J Pharmacol Exp Ther 261, 258-270

17. Lemoine, H. and Kaumann, A. J. (1982) Nauny-Schmiedeberg's Arch Pharmacol 320, 130-144

18. Foster, D. N., Min, B., Foster, L. K., Stoflet, E. S., Sun, S., Getz, M. J., and Strauch, A. R. (1992) J.Biol.Chem. 267, 11995-12003

19. McGraw, D. W. and Liggett, S. B. (1997) J.Biol.Chem. 272, 7338-7343

20. Wang, J., Niu, W., Nikiforov, Y., Naito, S., Chernausek, S., Witte, D., LeRoith, D., Strauch, A., and Fagin, J. A. (1997) J Clin Invest 100, 1425-1439

21. Green, S. A., Turki, J., Bejarano, P., Hall, I. P., and Liggett, S. B. (1995) i Am J Resp Cell Mol Biol 13, 25-33

22. D'Angelo, D. D., Sakata, Y., Lorenz, J. N., Boivin, G. P., Walsh, R. A., Liggett, S. B., and Dorn, G. W., II (1997) Proc.Natl.Acad.Sci., USA 94, 8121-8126

23. Green, S. and Liggett, S. B. (1994) J.Biol.Chem. 269, 26215-26219

24. Liggett, S. B., Bouvier, M., Hausdorff, W. P., O'Dowd, B., Caron, M. G., and Lefkowitz, R. J. (1989) Mol.Pharmacol. 36, 641-646

25. McGraw, D. W., Donnelly, E. T., Eason, M. G., Green, S. A., and Liggett, S. B. (1998) Cell Signal 10, 197-204

26. Kao, J., Fortner, C. N., Liu, L., Shull, G., and Paul, R. J. (1999) Am J Physiol:Lung Cell Mol Physiol (in press)

27. Sutliff, R. L. and Paul, R. J. (1998) in Cardiovascular Physiology in the Genetically Engineered Mouse (B. D. Hoit and R. A. Walsh, ed) pp. 247-257, Kluwer Academic, Norwell

28. Hamelmann, E., Schwarze, J., Takeda, K., Oshiba, A., Larsen, G. L., Irvin, C. G., and Gelfand, E. W. (1997) Am J Respir Crit Care Med 156, 766-775

29. Weibel, E. R. and Taylor, C. R. (1988) in Pulmonary Diseases and Disorders (Fishman, A. P., ed) pp. 20-21, McGraw-Hill, N.Y.

30. Laporte, J. D., Moore, P. E., Panettieri, R. A., Moeller, W., Heyder, J., and Shore, S. A. (1998) Am J Physiol 275, L91-L501

31. Turki, J., Lorenz, J. N., Green, S. A., Donnelly, E. T., Jacinto, M., and Liggett, S. B. (1996) Proc Natl Acad Sci, USA 93, 10483-10488

32. Milano, C. A., Allen, L. F., Rockman, H. A., Dolber, P. C., McMinn, T. R., Chien, K. R., Johnson, T. D., Bond, R. A., and Lefkowitz, R. J. (1994) Science 264, 582-586

33. Rockman, H. A., Chien, K. R., Choi, D. J., Iaccarino, G., Hunter, J. J., Ross, J. Jr., Lefkowitz, R. J., and Koch, W. J. (1998) Proc Natl Acad Sci USA 95, 7000-7005

34. Dorn, G. W. 2., Tepe, N. M., Lorenz, J. N., Koch, W. J., and Liggett, S. B. (1999) Proc Natl Acad Sci USA 96, 6400-6405

35. Milligan, G., Kim, G. D., Mullaney, I., and Adie, E. J. (1995) Mol Cell Biochem 149-150, 213-216

1. A method of treating airway obstructive disease comprising the administration to a subject in need of such treatment a therapeutically effective amount of an isolated nucleic acid molecule of the β₂AR gene or pharmaceutically acceptable composition thereof

2. The method of claim 1, wherein the airway obstructive disease is a respiratory disorder selected from the group consisting of airway obstruction, allergies, asthma, acute inflammatory lung disease, chronic inflammatory lung disease, chronic obstructive pulmonary dysplasia, emphysema, pulmonary emphysema, chronic obstructive emphysema, adult respiratory distress syndrome, bronchitis, chronic bronchitis, chronic asthmatic bronchitis, chronic obstructive bronchitis, and intestitial lung diseases.

3. The method of claim 2, wherein the nucleic acid molecule is a DNA molecule.

4. The method of claim 3, wherein the nucleic acid molecule is expressed in the subject's airway cells.

5. The method of claim 4, wherein the nucleic acid that encodes a β₂AR polypeptide, fragment, homolog or variant with substantial homology, supplying β₂AR function.

6. The method of claim 5, wherein the nucleic acid molecule becomes integrated to the chromosomal DNA making up the genome of the subject's airway cells.

7. The method of claim 5, wherein the nucleic acide molecule is expressed by the subject's airway cells from an extrachromosomal location.

8. The method of claim 5, wherein the nucleic acid molecule comprises at least 200 nucleotides.

9. The method of claim 5, wherein the nucleic acid molecule comprises at least 400 nucleotides.

10. The method of claim 5, wherein the airway cells are selected from the group consisting of smooth muscle and epithelial cells.

11. The method of claim 10, wherein the airway cells are selected from the group consisting of smooth muscle and epithelial cells.


SEQUENCE LISTING (4 sequences)

SEQ ID NO: 1 - DNA, 1242 bp, Homo sapiens
auggggcaacccgggaacggcagcgccuucuugcuggcacccaauagaagccaugcgccggaccacgacgucacgcagcaaagggacgagguguggguggugggcaugggcaucgucaugucucucaucguccuggccaucguguuuggcaaugugcuggucaucacagccauugccaaguucgagcgucugcagacggucaccaacuacuucaucacuucacuggccugugcugaucuggucaugggccuggcaguggugcccuuuggggccgcccauauucuuaugaaaauguggacuuuuggcaacuucuggugcgaguuuuggacuuccauugaugugcugugcgucacggccagcauugagacccugugcgugaucgcaguggaucgcuacuuugccauuacuucaccuuucaaguaccagagccugcugaccaagaauaaggcccgggugaucauucugaugguguggauugugucaggccuuaccuccuucuugcccauucagaugcacugguaccgggccacccaccaggaagccaucaacugcuaugccaaugagaccugcugugacuucuucacgaaccaagccuaugccauugccucuuccaucguguccuucuacguuccccuggugaucauggucuucgucuacuccagggucuuucaggaggccaaaaggcagcuccagaagauugacaaaucugagggccgcuuccauguccagaaccuuagccagguggagcaggaugggcggacggggcauggacuccgcagaucuuccaaguucugcuugaaggagcacaaagcccucaagacguuaggcaucaucaugggcacuuucacccucugcuggcugcccuucuucaucguuaacauugugcaugugauccaggauaaccucauccguaaggaaguuuacauccuccuaaauuggauaggcuaugucaauucugguuucaauccccuuaucuacugccggagcccagauuucaggauugccuuccaggagcuucugugccugcgcaggucuucuuugaaggccuaugggaauggcuacuccagcaacggcaacacaggggagcagaguggauaucacguggaacaggagaaagaaaauaaacugcugugugaagaccucccaggcacggaagacuuugugggccaucaagguacugugccuagcgauaacauugauucacaagggaggaauuguaguacaaaugacucacugcuguaa

SEQ ID NO: 2 - protein, 413 aa, Homo sapiens
  MISC_FEATURE (1)..(77)
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 3 - DNA, 20 bp, Homo sapiens
ggagcagagtggatatcacg

SEQ ID NO: 4 - DNA, 20 bp, Homo sapiens
gtcacaccacagaagtaagg